SEQUENCE LISTING (95 sequences)

SEQ ID NO: 1 - protein, 10 aa, Homo Sapiens
ALGGHPLLGV

SEQ ID NO: 2 - protein, 10 aa, Homo Sapiens
YLGGHPLLGV

SEQ ID NO: 3 - protein, 9 aa, Homo Sapiens
ILYPGGNKY

SEQ ID NO: 4 - protein, 9 aa, Homo Sapiens
ILYPGGNKV

SEQ ID NO: 5 - protein, 9 aa, Homo Sapiens
GILGFVFTL

SEQ ID NO: 6 - protein, 9 aa, Homo Sapiens
ILKEPVHGV

SEQ ID NO: 7 - protein, 10 aa, Homo Sapiens
VLNSNAIKNL

SEQ ID NO: 8 - protein, 10 aa, Homo Sapiens
VLNSNAIKNV

SEQ ID NO: 9 - protein, 10 aa, Homo Sapiens
ALGAAGATRV

SEQ ID NO: 10 - protein, 10 aa, Homo Sapiens
YLGAAGATRV

SEQ ID NO: 11 - protein, 9 aa, Homo Sapiens
AMCCPGNYV

SEQ ID NO: 12 - protein, 9 aa, Homo Sapiens
TLSSKMYHV

SEQ ID NO: 13 - protein, 10 aa, Homo Sapiens
YLNSVLNSNV

SEQ ID NO: 14 - protein, 9 aa, Homo Sapiens
RVFVAMVAA

SEQ ID NO: 15 - protein, 9 aa, Homo Sapiens
YLLGVSATL

SEQ ID NO: 16 - protein, 10 aa, Homo Sapiens
KMYHTKGQEG

SEQ ID NO: 17 - protein, 9 aa, Homo Sapiens
ILYPGGNKV

SEQ ID NO: 18 - protein, 9 aa, Homo Sapiens
YVLNSNAIV

SEQ ID NO: 19 - protein, 9 aa, Homo Sapiens
GLEIFQRCY

SEQ ID NO: 20 - protein, 9 aa, Homo Sapiens
YVFVAMVAV

SEQ ID NO: 21 - protein, 9 aa, Homo Sapiens
FVAMVAAAL

SEQ ID NO: 22 - protein, 10 aa, Homo Sapiens
VFVAMVAAAL

SEQ ID NO: 23 - protein, 10 aa, Homo Sapiens
HPLLGVSATL

SEQ ID NO: 24 - protein, 10 aa, Homo Sapiens
VLKEGQVCTK

SEQ ID NO: 25 - protein, 10 aa, Homo Sapiens
AMCCPGNYCK

SEQ ID NO: 26 - protein, 10 aa, Homo Sapiens
CVSSDQNHFR

SEQ ID NO: 27 - protein, 10 aa, Homo Sapiens
GVQICLACRK

SEQ ID NO: 28 - protein, 10 aa, Homo Sapiens
GVQICLACRK

SEQ ID NO: 29 - protein, 9 aa, Homo Sapiens
SVLNSNAIK

SEQ ID NO: 30 - protein, 10 aa, Homo Sapiens
CVSSDQNHFR

SEQ ID NO: 31 - protein, 10 aa, Homo Sapiens
CYCGEGLSCR

SEQ ID NO: 32 - protein, 9 aa, Homo Sapiens
EIEETITER

SEQ ID NO: 33 - protein, 10 aa, Homo Sapiens
CVSSDQNHFR

SEQ ID NO: 34 - protein, 9 aa, Homo Sapiens
DGYSRRTTL

SEQ ID NO: 35 - protein, 9 aa, Homo Sapiens
CRKRRKRCM

SEQ ID NO: 36 - protein, 10 aa, Homo Sapiens
GEIEETITES

SEQ ID NO: 37 - protein, 10 aa, Homo Sapiens
EECGTDEYCA

SEQ ID NO: 38 - protein, 9 aa, Homo Sapiens
GDAGVQICL

SEQ ID NO: 39 - protein, 10 aa, Homo Sapiens
LDGYSRRTTL

SEQ ID NO: 40 - protein, 10 aa, Homo Sapiens
EECGTDEYCA

SEQ ID NO: 41 - protein, 10 aa, Homo Sapiens
GDAGVQICLA

SEQ ID NO: 42 - protein, 9 aa, Homo Sapiens
ILYPGGNKY

SEQ ID NO: 43 - protein, 10 aa, Homo Sapiens
YQTIDNYQPY

SEQ ID NO: 44 - protein, 10 aa, Homo Sapiens
HPLLGVSATL

SEQ ID NO: 45 - protein, 9 aa, Homo Sapiens
AVSAAPGIL

SEQ ID NO: 46 - protein, 10 aa, Homo Sapiens
ACRKRRKRCM

SEQ ID NO: 47 - protein, 10 aa, Homo Sapiens
SCRIQKDHHV

SEQ ID NO: 48 - protein, 10 aa, Homo Sapiens
RRTTLSSKMY

SEQ ID NO: 49 - protein, 10 aa, Homo Sapiens
RRKGSHGLEI

SEQ ID NO: 50 - protein, 9 aa, Homo Sapiens
HRRKGSHGL

SEQ ID NO: 51 - protein, 9 aa, Homo Sapiens
SRRTTLSSK

SEQ ID NO: 52 - protein, 9 aa, Homo Sapiens
RSSDCASGL

SEQ ID NO: 53 - protein, 9 aa, Homo Sapiens
WSKICKPVL

SEQ ID NO: 54 - protein, 9 aa, Homo Sapiens
GDAGVQICL

SEQ ID NO: 55 - protein, 10 aa, Homo Sapiens
SDQNHFRGEI

SEQ ID NO: 56 - protein, 10 aa, Homo Sapiens
HHQASNSSRL

SEQ ID NO: 57 - protein, 10 aa, Homo Sapiens
KHRRKGSHGL

SEQ ID NO: 58 - protein, 10 aa, Homo Sapiens
HHQASNSSRL

SEQ ID NO: 59 - protein, 10 aa, Homo Sapiens
KHRRKGSHGL

SEQ ID NO: 60 - protein, 9 aa, Homo Sapiens
HQASNSSRL

SEQ ID NO: 61 - protein, 10 aa, Homo Sapiens
TKGQEGSVCL

SEQ ID NO: 62 - protein, 9 aa, Homo Sapiens
DEECGTDEY

SEQ ID NO: 63 - protein, 9 aa, Homo Sapiens
IEETITESF

SEQ ID NO: 64 - protein, 10 aa, Homo Sapiens
YPGGNKYQTI

SEQ ID NO: 65 - protein, 10 aa, Homo Sapiens
CPGNYCKNGI

SEQ ID NO: 66 - protein, 10 aa, Homo Sapiens
YPGGNKYQTI

SEQ ID NO: 67 - protein, 10 aa, Homo Sapiens
CPGNYCKNGI

SEQ ID NO: 68 - protein, 10 aa, Homo Sapiens
GAAGATRVFV

SEQ ID NO: 69 - protein, 10 aa, Homo Sapiens
GAAGHPGSAV

SEQ ID NO: 70 - protein, 10 aa, Homo Sapiens
LGAAGATRVF

SEQ ID NO: 71 - protein, 9 aa, Homo Sapiens
KGSHGLEIF

SEQ ID NO: 72 - protein, 10 aa, Homo Sapiens
ASGLCCARHF

SEQ ID NO: 73 - protein, 9 aa, Homo Sapiens
GAAGATRVF

SEQ ID NO: 74 - protein, 10 aa, Homo Sapiens
NAIKNLPPPL

SEQ ID NO: 75 - protein, 10 aa, Homo Sapiens
SAVSAAPGIL

SEQ ID NO: 76 - protein, 10 aa, Homo Sapiens
VFVAMVAAAL

SEQ ID NO: 77 - protein, 10 aa, Homo Sapiens
HPLLGVSATL

SEQ ID NO: 78 - protein, 10 aa, Homo Sapiens
NAIKNLPPPL

SEQ ID NO: 79 - protein, 10 aa, Homo Sapiens
GATRVFVAMV

SEQ ID NO: 80 - protein, 9 aa, Homo Sapiens
NDHSTLDGY

SEQ ID NO: 81 - protein, 9 aa, Homo Sapiens
QTIDNYQPY

SEQ ID NO: 82 - protein, 15 aa, Homo Sapiens
TRVFVAMVAAALGGH

SEQ ID NO: 83 - protein, 15 aa, Homo Sapiens
MMALGAAGATRVFVA

SEQ ID NO: 84 - protein, 15 aa, Homo Sapiens
GICVSSDQNHFRGEI

SEQ ID NO: 85 - protein, 15 aa, Homo Sapiens
LLGVSATLNSVLNSN

SEQ ID NO: 86 - protein, 15 aa, Homo Sapiens
SATLNSVLNSNAIKN

SEQ ID NO: 87 - protein, 15 aa, Homo Sapiens
ASGLCCARHFWSKIC

SEQ ID NO: 88 - protein, 15 aa, Homo Sapiens
LLGVSATLNSVLNSN

SEQ ID NO: 89 - protein, 15 aa, Homo Sapiens
RGEIEETITESFGND

SEQ ID NO: 90 - protein, 15 aa, Homo Sapiens
RHFWSKICKPVLKEG

SEQ ID NO: 91 - protein, 15 aa, Homo Sapiens
GNKYQTIDNYQPYPC

SEQ ID NO: 92 - protein, 15 aa, Homo Sapiens
YQTIDNYQPYPCAED

SEQ ID NO: 93 - protein, 15 aa, Homo Sapiens
AGATRVFVAMVAAAL

SEQ ID NO: 94 - DNA, 801 bp, Homo Sapiens
atgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaa

SEQ ID NO: 95 - protein, 266 aa, Homo Sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

(19) United States
(12) Patent Application Publication
(10) Pub. No.: US 2010/0129390 A1
(43) Pub. Date: May 27, 2010

(54) DKK1 AS A UNIVERSAL TUMOR VACCINE FOR IMMUNOTHERAPY OF CANCERS

(76) Inventors: Qing Yi, Pearland, TX (US); Jianfei Qian, Houston, TX (US)

Correspondence Address: Nielsen IP Law LLC, 1177 West Loop South, Suite 1600, Houston, TX, 77027, US

(21) Appl. No.: 12/620,144

(22) Filed: Nov. 17, 2009

Related U.S. Application Data
(63) Continuation-in-part of application No. PCT/US08/64138, filed on May 19, 2008.
(60) Provisional application No. 60/938,955, filed on May 18, 2007.

(51) Int. Cl. A61K 39/00; C07K 7/06; A61K 35/14; A61P 35/00; A61P 13/08; C12N 5/07

(52) U.S. Cl. 424/185.1; 530/328; 424/184.1; 424/93.71; 435/375

(57) ABSTRACT

DKK1 compositions comprising a DKK1 protein, DKK1 peptide, DKK1 DNA, DKK1-specific CTLs and Th1 cells and associated methods for treating cancer and cancer-mediated disorders in a human or animal subject in need of such treatment by administering a DKK1 composition to prevent or treat cancer are disclosed herein. The DKK1 compositions can be used alone, or in combination with other treatments, for the treatment of various cancers including ovarian, breast, colonic, brain, lung, prostate, pancreatic, lymphoma, esophageal carcinomas and melanoma.

### CROSS-REFERENCE TO RELATED APPLICATIONS

This application is a continuation-in-part of International Application No. PCT/US2008/064138 filed May 19, 2008 which claims priority to U.S. Patent. App. Ser. No. 60/938,955 filed May 18, 2007, both of which are incorporated herein by reference.

### STATEMENT REGARDING FEDERALLY SPONSORED RESEARCH OR DEVELOPMENT

None.

### REFERENCE TO SEQUENCE LISTING

The Sequence Listing is contained on an electronic text file named Sequence Listing.txt which is 18.2 kilobytes in size and was created on Nov. 17, 2009. The material contained in the .txt file is being filed concurrently via USPTO EFS-Web with the present specification and is hereby incorporated-by-reference.

### BACKGROUND

Dickkopf-1 (herein also referred to as “DKK1” and sometimes spelled by others as “Dikkopf-1”) is a secreted protein that specifically inhibits Wnt/β-catenin signaling by interacting with the co-receptor Lrp-6. Mao, B., et al., LDL-Receptor-Related Protein 6 is a Receptor for Dickkopf Proteins, Nature. 2001, 411:321-325; Zorn, A. M., Wnt Signaling: Antagonistic Dickkopfs, Curr Biol. 2001, 11:R592-595. Recent studies demonstrate that DKK1 in myeloma patients is associated with the presence of lytic bone lesions. Tian, E., et al., The Role of the Wnt-Signaling Antagonist DKK1 in the Development of Osteolytic Lesions in Multiple Myeloma, N Engl J. Med. 2003, 349:2483-2494. It has been shown that blocking DKK1 activity may reduce osteolytic bone resorption, increase bone formation, and help control myeloma progression. Yaccoby, S., et al., Antibody-Based Inhibition of DKK1 Suppresses Tumor-Induced Bone Resorption and Multiple Myeloma Growth In-Vivo, Blood. 2006. Studies have also shown that the DKK1 gene has a restricted expression in placenta and mesenchymal stem cells (MSCs) only and not in other normal tissues. Glinka, A., et al., Dickkopf-1 is a Member of a New Family of Secreted Proteins and Functions in Head Induction, Nature. 1998, 391:357-362; Gregory, C. A., et al., The Wnt Signaling Inhibitor Dickkopf-1 is Required for Reentry Into the Cell Cycle of Human Adult Stem Cells From Bone Marrow, J Biol. Chem. 2003, 278:28067-28078. Furthermore, a gene expression profile analysis of lung and esophageal carcinomas revealed that DKK1 is highly transactivated in the great majority of lung cancers and esophageal squamous cell carcinomas (ESCC). Yamabuki, T. et al, Dikkopf-1 as a Novel Serologic and Prognostic Biomaker for lung and Esophageal Carcinomas, Cancer Res. 2007, 67(6): 2517-25.

Multiple myeloma (herein also referred to as “MM”) is a fatal hematological malignancy characterized by the accumulation of terminally differentiated plasma cells in the bone marrow of patients. Tricot, G., et al., Graft-Versus-Myeloma Effect: Proof of Principle, Blood. 1996, 87:1196-1198. The outcome of the majority of patients with MM is unsatisfactory, although may patients do benefit from high-dose therapy followed by autologous stem cell support. Blade, J., et al., High-Dose Therapy in Multiple Myeloma, Blood. 2003, 102:3469-3470. Immunotherapy may be an alternative appropriate means to control residual disease as well as to provide an alternative treatment modality to conventional chemotherapy for patients with MM and other cancers. Clearly, there is a need for new cancer treatments including those that stabilize or even eradicate minimal residual disease achieved after the treatment with high-dose chemotherapy and stem-cell transplantation.

### SUMMARY

The present disclosure relates generally to Dickkopf-1, and more specifically relates to DKK1 compositions and associated methods for treating cancer and cancer-mediated disorders.

DKK1 compositions comprising a DKK1 protein, DKK1 peptide, DKK1 DNA, DKK1-specific CTLs and/or DKK1-specific Th1 cells and associated methods for treating cancer and cancer-mediated disorders are disclosed herein. The DKK1 compositions can be used alone, or in combination with other treatments, for the treatment of various cancers including ovarian, breast, colonic, brain, lung, prostate, pancreatic, lymphoma, esophageal carcinomas and melanoma.

Novel antigens (DKK1 protein, DKK1 peptides, or DKK1 DNA) presented on both class I and class II major histocompatibility molecules (“MHC”) useful in stimulating anti-cancer CD8⁺ cytotoxic T lymphocytes (“CTLs”) and CD4⁺ type-1 helper T cells (Th1) and antibody responses are provided. Vaccines comprising DKK1 protein, DKK1 peptides, or DKK1 DNA can be used to generate DKK1-specific CTLs and Th1 cells useful as effector cells and to induce DKK1-specific antibodies in cancer treatments. A DKK1 peptide, DKK1 protein, or DKK1 DNA, alone or in combination with other DKK1 peptides or immunoadjuvants can be used as vaccines to prevent or treat cancer. Similarly, DKK1-specific CTLs and Th1 cells are useful alone or in combination with one or more DKK1 peptides or the DKK1 protein together with other therapeutic agents for cancer treatment. The methods of making DKK1 peptides, DKK1 protein or DKK1 DNA including a novel host cell and vector construct are also described. Methods for inducing an immune response against cancer in a subject in need thereof are also provided. Methods of treating cancer by administering a DKK1 composition are provided.

### BRIEF DESCRIPTION OF THE DRAWINGS

The foregoing summary as well as the following detailed description will be better understood when read in conjunction with the appended drawings. It should be understood, however, that the disclosure is not limited to the precise arrangements and instrumentalities shown herein.

FIG. 1A depicts the lack of mRNA for DKK1 in most human tissues.

FIG. 1B depicts the detection of DKK1 mRNA in 8 myeloma cell lines.

FIG. 1C depicts the detection of DKK1 protein in 7 out of 10 myeloma cell lines and all primary myeloma cells of 10 multiple melanoma patients.

FIG. 2A shows the affinity binding for two control peptides and four peptides SEQ ID NOS 1-6.

FIG. 2B shows peptide binding time for certain DKK1 peptides SEQ ID NOS 1-4.

FIG. 2C shows the tests results for peptide binding detected by IFN-γ expression.

FIG. 2D shows peptide (P20 or P66v)-HLA-A*0201 tetramer⁺ CD8⁺ T cells expression.

FIG. 2E evidences strong cytolytic activity against peptide pulsed, but not unpulsed, T2 cells.

FIG. 3 shows that DKK1 peptide-specific CD⁺8 T cells appear in lower frequency in healthy donors opposed to myeloma patients.

FIG. 4A shows the number of T-cell lines proliferated in response to autologous dendritic cells pulsed and unpulsed with the DKK1 peptides.

FIG. 4B the number of CTLs proliferated in response to autologous dendritic cells pulsed and unpulsed with the DKK1 peptides.

FIG. 4C show that the same results depicted in FIGS. 4A and 4B can be shown with CFSE-labeling assay.

FIG. 4D shows that CTLs increase during in vitro stimulation with the peptides.

FIG. 4E shows the cytotoxicity of DKK1 peptide-specific CTLs.

FIG. 4F shows the cytotoxicity of DKK1 peptide-specific CTLs.

FIG. 5A shows the existence of DKK1 peptide-specific CTL clones generated with peptides disclosed herein.

FIG. 5B shows the existence of DKK1 peptide-specific CTL clones generated with the peptides disclosed herein.

FIG. 5C shows the cytolytic activity of the DKK1 peptide-specific CTL clones generated with the peptides.

FIG. 5D shows DKK1 peptide-specific CTL clones generated with the peptides of the subject disclosure lysed T2 pulsed with the specific DKK1 peptide.

FIG. 6A shows DKK1 peptide-specific CTL clones generated with the peptides of the subject disclosure lysed U266 and IM-9 cells.

FIG. 6B shows DKK1 peptide-specific CTL clones generated with the peptides of the subject disclosure lysed U266 and IM-9 cells.

FIG. 6C shows DKK1 peptide-specific CTL clones generated with the peptides of the subject disclosure kill primary myeloma cells taken from patients having a particular MHC class molecule but not another.

FIG. 6D shows the inhibitory effect of anti-MHC mAbs.

FIG. 7A shows that the CTL clones of the present disclosure kill their target cells via the perforin/granzyme pathways.

FIG. 7B shows a representative experiment of intracellular cytokine staining for IFN-γ and IL-4 expression in the P66v-specific T-cell clone.

FIG. 7C shows large numbers of IFN-γ-secreting cells were detected after re-stimulation with dendritic cells pulsed with DKK1 peptides.

FIG. 8A shows the effects of adoptively transferred DKK1 peptide-specific CTLs in SCID-hu mice. Shown are the changes of tumor burdens in SCID-hu mice (3 per group) receiving injections of 5×10⁶ DKK-specific CTLs or CD8⁺ T cells. Control mice received injections of PBS (untreatment).

FIG. 8B shows the effects of adoptively transferred DKK1 peptide-specific CTLs on myeloma cells in vivo. Shown are the changes of tumor burdens, measured as the levels of circulating human M-protein in SCID-hu mice (3 per group) receiving injections of 5×10⁶ DKK-specific CTLs or CD8⁺ T cells. Control mice received injections of PBS (untreatment). Arrows indicate injection of the T cells.

FIG. 9A provides the DKK1 DNA sequence.

FIG. 9B provides the human DKK1 protein sequence.

FIG. 10 shows one embodiment of a DKK1 DNA vaccine construct useful to produce DKK1 peptide-specific CTLs described herein.

FIG. 11A depicts the detection of DKK1 mRNA in 6 out of 10 lymphoma cell lines, 3 breast cancer cell lines, and 2 out of 3 prostate cancer cell lines.

FIG. 11B depicts the detection of DKK1 protein in 6 out of 10 lymphoma cell lines, 3 breast cancer cell lines, and 2 out of 3 prostate cancer cell lines.

FIGS. 12A and 12B show the cytotoxicity of DKK1 peptide-specific CTLs in lymphoma cell lines.

FIGS. 13A and 13B show the cytotoxicity of DKK1 peptide-specific CTLs in breast cancer cell lines.

FIGS. 14A and 14B show the cytotoxicity of DKK1 peptide-specific CTLs in prostate cancer cell lines.

FIG. 15 shows the effects of adoptively transferred DKK1 peptide-specific CTLs on lymphoma cells in vivo.

FIG. 16 shows the effects of adoptively transferred DKK1 peptide-specific CTLs on prostate cancer cells in vivo.

FIG. 17A depicts DKK1 gene expression in some primary mantle cell lymphoma (ML4, ML5 and ML6) and follicular lymphoma cells (FL5).

FIG. 17B depicts western blots showing DKK1 protein expression in expression in some of primary mantle cell lymphoma (ML4, ML5 and ML6) and follicular lymphoma cells (FL5).

FIG. 18A is a graph depicting ELISA titers of anti-DKK1 antibody in the serum of immunized mice.

FIG. 18B is a graph depicting the survival of mice that were innoculated with a DKK1 protein vaccination and PBS control.

FIG. 18C is a graph depicting the cytotoxicity of CTLs against murine myeloma cell lines tumor-A, B, C and D.

FIG. 19A is a graph depicting the survival of mice that were innoculated with a DKK1 peptide vaccination and PBS control.

FIG. 19B is a graph depicting the cytotoxicity of CTLs against murine myeloma cell lines tumor-A, B, C and D.

FIG. 20A is a graph depicting ELISA titers of anti-DKK1 antibody in the serum of immunized mice.

FIG. 20B is a graph depicting the survival of mice that were innoculated with a DKK1 DNA vaccination and PBS control.

FIG. 20C is a graph depicting the cytotoxicity of CTLs against murine myeloma cell lines tumor-A, B, C and D.

FIG. 21 depicts intracellular staining for CD4⁺ IFN-γ or IL-4-expressing T cells.

FIG. 22 intracellular staining for CD8⁺ IFN-γ or IL-4-expressing T cells.

### DETAILED DESCRIPTION

The present disclosure provides a DKK1-based immunotherapy in multiple melanoma, B-cell lymphomas and other cancers. DKK1 is a potent tumor-associated antigen (TAA) in MM and other cancers. In one embodiment, the present disclosure provides a DKK1 vaccine that comprises a DKK1 protein, a DKK1 peptide and/or DKK1 DNA. In some embodiments, a DKK1 vaccine may be administered to a patient to generate DKK1-specific cytotoxic T lymphocytes (CTLs), Th1 cells, and/or antibodies in the patients. Also, in some embodiments, DKK1-specific CTLs and Th1 cells may be administered to a patient and used as effector cells for immunotherapy in cancer, including multiple melanoma, B-cell lymphomas, breast and prostate cancers.

As noted above, Dickkopf-1 (DKK1) is a secreted protein that specifically inhibits the Wnt/β-catenin signaling by interacting with the co-receptor Lrp-6. Previous studies have shown that the DKK1 gene has a restricted expression in placenta and mesenchymal stem cells (MSCs) only and not in other normal tissues. Glinka, A., et al., Dickkopf-1 is a Member of a New Family of Secreted Proteins and Functions in Head Induction, Nature. 1998, 391:357-362; Gregory, C. A., et al., The Wnt Signaling Inhibitor Dickkopf-1 is Required for Reentry Into the Cell Cycle of Human Adult Stem Cells From Bone Marrow, J Biol. Chem. 2003, 278:28067-28078. Recent studies have demonstrated that DKK1 in myeloma patients is associated with the presence of lytic bone lesions. Tian, E., et al., The Role of the Wnt-Signaling Antagonist DKK1 in the Development of Osteolytic Lesions in Multiple Myeloma, N Engl J. Med. 2003, 349:2483-2494. Immunohistochemical analysis of bone marrow biopsy specimens also showed that only myeloma cells contain detectable DKK1. It has also been shown that recombinant human DKK1 or bone marrow serum containing an elevated level of DKK1 inhibited the differentiation of osteoblast precursor cells in vitro. Furthermore, anti-DKK1 antibody treatment is associated with reduced tumor growth in a myeloma mouse model. These results indicate that DKK1 is an important player in myeloma bone disease and blocking DKK1 activity reduced osteolytic bone resorption, increased bone formation, and helped control myeloma progression. Yaccoby, S., et al., Antibody-Based Inhibition of DKK1 Suppresses Tumor-Induced Bone Resorption and Multiple Myeloma Growth In-Vivo, Blood. 2006.

As previously mentioned above, the present disclosure relates generally to DKK1 proteins, DKK1 peptides, DKK1 DNA, DKK1-specific CTLs and DKK1-specific Th1 cells, collectively referred to herein as “DKK1 compositions.” In one embodiment, the present disclosure provides a DKK1 vaccine that may comprise a DKK1 protein, a DKK1 peptide and/or DKK1 DNA. Examples of suitable DKK1 peptides include DKK1 peptides for class I major histocompatability molecules including HLA-A, HLA-B and HLA-C and class II major histocompatability molecules. DKK1 peptides suitable for use in the present disclosure include, but are not limited to, those DKK1 peptides provided in Table I, Table II and Table III below.

TABLE I
HLA-A*201 Molecules

| SEQ | | | | Predictive |
|---|---|---|---|---|
| ID | | | | binding |
| NO. | Name | Sequence | Position | score^{a} |
| 1 | P20 | ALGGHPLLGV | 20 | 160 |
| 2 | Py20 | YLGGHPLLGV | 20 | 736 |
| 3 | P66 | ILYPGGNKY | 66 | 0.4 |
| 4 | P66v | ILYPGGNKV | 66 | 378 |
| 5 | Flu matrix | GILGFVFTL | 58 | 550 |
| 6 | HIV pol | ILKEPVHGV | 476 | 39 |
| 7 | P36 | VLNSNAIKNL | 36 | 84 |
| 8 | P36v | VLNSNAIKNV | 36 | 226 |
| 9 | P3 | ALGAAGATRV | 3 | 70 |
| 10 | Py3 | YLGAAGATRV | 3 | 320 |
| 11 | P125v | AMCCPGNYV | 125 | 276 |
| 12 | P173v | TLSSKMYHV | 173 | 160 |
| 13 | Py32v | YLNSVLNSNV | 32 | 320 |
| 14 | Py11v | RVFVAMVAA | 11 | 238 |
| 15 | Py25 | YLLGVSATL | 25 | 364 |
| 16 | P177v | KMYHTKGQEG | 177 | 562 |
| 17 | P66v | ILYPGGNKV | 66 | 378 |

TABLE II
DKK1 peptides for Other MHC Class I Molecules

| SEQ | | | | | Predictive |
|---|---|---|---|---|---|
| ID | | | | | binding |
| NO. | HLA | Name | Sequence | Position | score^{a} |
| 18 | A1 | Py35v | YVLNSNAIV | 35 | 42.5 |
| 19 | A1 | P230 | GLEIFQRCY | 230 | 45 |
| 20 | A0205 | Py11v | YVFVAMVAV | 11 | 72 |
| 21 | A0205 | P13 | FVAMVAAAL | 13 | 42 |
| 22 | A24 | P12 | VFVAmVAAAL | 12 | 42 |
| 23 | A24 | P24 | HPLLgVSATL | 24 | 8.4 |
| 24 | A3 | P213 | VLKEgQVCTK | 213 | 90 |
| 25 | A3 | P125 | AMCCpGNYCK | 125 | 60 |
| 26 | A68.1 | P138 | CVSSdQNHFR | 138 | 200 |
| 27 | A68.1 | P107 | GVQIcLACRK | 107 | 120 |
| 28 | A1101 | P107 | GVQIcLACRK | 107 | 6 |
| 29 | A1101 | P35 | SVLNSNAIK | 35 | 3 |
| 30 | A3101 | P138 | CVSSdQNHFR | 138 | 2 |
| 31 | A3101 | P237 | CYCGeGLSCR | 237 | 1.2 |
| 32 | A3302 | P149r | EIEETITER | 149 | 45 |
| 33 | A3302 | P138 | CVSSdQNHFR | 138 | 15 |
| 34 | B14 | P166 | DGYSRRTTL | 166 | 1125 |
| 35 | B14 | P114 | CRKRRKRCM | 114 | 40 |
| 36 | B40 | P148 | GEIEeTITES | 148 | 32 |
| 37 | B40 | P89 | EECGtDEYCA | 89 | 20 |
| 38 | B60 | P104 | GDAGVQICL | 104 | 88 |
| 39 | B60 | P165 | LDGYsRRTTL | 165 | 20 |
| 40 | B61 | P89 | EECGtDEYCA | 89 | 30 |
| 41 | B61 | P104 | GDAGvQICLA | 104 | 11 |
| 42 | B62 | P66 | ILYPGGNKY | 66 | 124.8 |
| 43 | B62 | P74 | YQTIdNYQPY | 74 | 80 |
| 44 | B7 | P24 | HPLLgVSATL | 24 | 80 |
| 45 | B7 | P59 | AVSAAPGIL | 59 | 60 |
| 46 | B8 | P113 | ACRKrRKRCM | 113 | 80 |
| 47 | B8 | P244v | SCRIqKDHHV | 244 | 48 |
| 48 | B2702 | P170 | RRTTISSKMY | 170 | 180 |
| 49 | B2702 | P224 | RRKGsHGLEI | 224 | 180 |
| 50 | B2705 | P23 | HRRKGSHGL | 223 | 2000 |
| 51 | B2705 | P169 | SRRTTLSSK | 169 | 2000 |
| 52 | B3501 | P191 | RSSDCASGL | 191 | 20 |
| 53 | B3501 | P206 | WSKICKPVL | 206 | 15 |
| 54 | B3701 | P104 | GDAGVQICL | 104 | 60 |
| 55 | B3701 | P141 | SDQNhFRGEI | 141 | 30 |
| 56 | B3801 | P251 | HHQAsNSSRL | 251 | 30 |
| 57 | B3801 | P222 | KHRRkGSHGL | 222 | 9 |
| 58 | B3901 | P251 | HHQAsNSSRL | 251 | 90 |
| 59 | B3901 | P222 | KHRRkGSHGL | 222 | 27 |
| 60 | B3902 | P252 | HQASNSSRL | 252 | 20 |
| 61 | B3902 | P181 | TKGQeGSVCL | 181 | 20 |
| 62 | B4403 | P88 | DEECGTDEY | 88 | 540 |
| 63 | B4403 | P150 | IEETITESF | 150 | 60 |
| 64 | B5101 | P68 | YPGGnKYQTI | 68 | 692 |
| 65 | B5101 | P128 | CPGNyCKNGI | 128 | 440 |
| 66 | B5102 | P68 | YPGGnKYQTI | 68 | 1064.8 |
| 67 | B5102 | P128 | CPGNyCKNGI | 128 | 440 |
| 68 | B5103 | P121 | GAAGaTRVFV | 5 | 121 |
| 69 | B5103 | P51 | GAAGhPGSAV | 51 | 110 |
| 70 | B5201 | P4 | LGAAgATRVF | 4 | 74.3 |
| 71 | B5201 | P226 | KGSHGLEIF | 226 | 45.4 |
| 72 | B5801 | P196 | ASGLcCARHF | 196 | 40 |
| 73 | B5801 | P5 | GAAGATRVF | 5 | 20 |
| 74 | CW0301 | P40 | NAIKnLPPPL | 40 | 40 |
| 75 | CW0301 | P58 | SAVSaAPGIL | 58 | 40 |
| 76 | CW0401 | P12 | VFVAmVAAAL | 12 | 240 |
| 77 | CW0401 | P24 | HPLLgVSATL | 24 | 96 |
| 78 | CW0602 | P40 | NAIKnLPPPL | 40 | 6.6 |
| 79 | CW0602 | P8 | GATRvFVAMV | 8 | 6.6 |
| 80 | CW0702 | P160 | NDHSTLDGY | 160 | 16 |
| 81 | CW0702 | P75 | QTIDNYQPY | 75 | 11.2 |

TABLE III
DKK1 peptides for MHC class II molecules

| SEQ | | | | Po- | SYFPEITHI |
|---|---|---|---|---|---|
| ID | | | | si- | Predictive |
| NO. | HLA | Name | Sequence | tion | score |
| 82 | DRB1*0101 | P10 | TRVFVAMVAAALGGH | 10 | 33 |
| 83 | DRB1*0101 | P1 | MMALGAAGATRVFVA | 1 | 26 |
| 84 | DRB1*0301 | P136 | GICVSSDQNHFRGEI | 136 | 27 |
| 85 | DRB1*0301 | P26 | LLGVSATLNSVLNSN | 26 | 25 |
| 86 | DRB1*0401 | P30 | SATLNSVLNSNAIKN | 30 | 26 |
| 87 | DRB1*0401 | P196 | ASGLCCARHFWSKIC | 196 | 26 |
| 88 | DRB1*0701 | P26 | LLGVSATLNSVLNSN | 26 | 30 |
| 89 | DRB1*0701 | P147 | RGEIEETITESFGND | 147 | 30 |
| 90 | DRB1*1101 | P203 | RHFWSKICKPVLKEG | 203 | 25 |
| 91 | DRB1*1l01 | P71 | GNKYQTIDNYQPYPC | 71 | 22 |
| 92 | DRB1*1501 | P74 | YQTIDNYQPYPCAED | 74 | 28 |
| 93 | DRB1*1501 | P7 | AGATRVFVAMVAAAL | 7 | 24 |

Table I represents DKK1 peptides for HLA-A*0201 molecules. Table II provides DKK1 peptides for other MHC class I molecules. Table III provides DKK1 peptides for MHC class II molecules. DKK1-specific cytotoxic T lymphocyte (“CTL”) lines and clones can be generated using the DKK1 protein, a DKK1 peptide, and/or DKK1 DNA described herein. These DKK1 peptides, alone or in combination with other DKK1 peptides or the DKK1 protein or DNA itself, are useful as universal vaccines to immunize patients.

Modifications can be made to the DKK1 protein, DKK1 peptide, and/or DKK1 DNA suitable for use in the present disclosure in order to improve its stability and/or biological activity. Such modifications may include, but are not limited to, N-terminal (acetylation, glycosylation) or C-terminal (amidation) modifications, the use of unnatural amino acids (e.g. β-amino and α-trifluoromethyl amino acids) particularly at labile sites, cyclization and coupling with carriers such as polyethylene glycol (PEG). A DKK1 protein, a DKK1 peptide or DKK1 DNA may be designed to generate an immune response in a subject and likewise is useful in a vaccine to prevent, inhibit, modulate and/or treat cancer and associated tumors.

In some embodiments, DKK1-specific CTLs and/or Th1 cells may be administered to a patient. As described herein, DKK1-specific CTLs have been shown to be potent killer cells able to specifically and effectively lyse cancer cells, including myeloma cells, lymphoma, breast, and prostate cancer cells, while not effecting normal blood cells. More specifically, DKK1 peptide-specific CTLs have been shown to have strong cytolytic activity against DKK1 peptide-pulsed T2 cells and autologous dendritic cells. More importantly, while DKK1-specific CTLs show strong cytolytic activity against primary myeloma cells, they are not cytolytic against patients not having that particular MHC molecule. DKK1-specific CTLs or Th1 cells either alone or in combination with one or more DKK1 peptides or the DKK1 protein itself are useful against cancer alone or in combination with other cancer treatments.

While it may be possible for a DKK1 composition to be administered as a raw composition, it is also possible to present them as a pharmaceutical formulation. Accordingly, a pharmaceutical formulation comprising a DKK1 composition or a pharmaceutically acceptable salt, ester, prodrug or solvate thereof, together with one or more pharmaceutically acceptable carriers thereof and optionally one or more other therapeutic ingredients is provided. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a peptide or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active peptide doses.

A DKK1 composition may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the peptides may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compositions may also be formulated in rectal compositions such as suppositories or retention enemas, e.g., containing conventional suppository bases such as cocoa butter, polyethylene glycol, or other glycerides.

DKK1 compositions described herein may be administered topically, that is by non-systemic administration. This includes the application of a DKK1 protein, a DKK1 peptide, or DKK1 DNA externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Preferred unit dosage formulations are those containing an effective dose, as herein below recited, or an appropriate fraction thereof, of the active ingredient.

It should be understood that in addition to the ingredients particularly mentioned above, the formulations may include other agents conventional in the art having regard to the type of formulation in question, for example those suitable for oral administration may include flavoring agents.

Preferred unit dosage formulations are those containing an effective dose, as herein below recited, or an appropriate fraction thereof, of the active ingredient. The compositions may be administered orally or via injection at a dose of from 0.1 to 500 mg/kg per day. The dose range for adult humans is generally from 5 mg to 2 g/day. Tablets or other forms of presentation provided in discrete units may conveniently contain an amount of peptide which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration. Hence, the phrase “therapeutically effective” is intended to qualify the amount of active ingredients used in the treatment of a disease or disorder. This amount will achieve the goal of reducing or eliminating the said disease or disorder. The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The DKK1 compositions can be administered in various modes, e.g. orally, topically, or by injection. The precise amount of DKK1 composition administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the peptides therein is hypertension, then it may be appropriate to administer an anti-hypertensive agent in combination with the initial therapeutic agent. Or, by way of example only, the therapeutic effectiveness of one of the peptides described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the peptides described herein with another therapeutic agent (which also includes a therapeutic regimen) that also has therapeutic benefit. By way of example only, in a treatment for cancer involving administration of one of the peptides described herein, increased therapeutic benefit may result by also providing the patient with another therapeutic agent for cancer. In any case, regardless of the disease, disorder or condition being treated, the overall benefit experienced by the patient may simply be additive of the two therapeutic agents or the patient may experience a synergistic benefit.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, methods for treating cancer and cancer-mediated disorders in a human or animal subject in need of such treatment are provided that comprise administering to said subject a therapeutically effective amount of a DKK1 composition effective to reduce or prevent said disorder in the subject in combination with at least one additional agent for the treatment of said disorder that is known in the art. In a related aspect, therapeutic compositions comprising at least one DKK1 composition in combination with one or more additional agents for the treatment of cancer are provided. Therapeutic compositions may further comprise an antibody in combination with one or more additional agents for the treatment of cancer.

The DKK1 compositions can be used alone, or in combination with other treatments, for the treatment of various cancers including ovarian, breast, colonic, brain, lung, prostate, pancreatic, lymphoma, esophageal carcinomas and melanoma.

Besides being useful for human treatment, the DKK1 compositions are also useful for veterinary treatment of companion animals, exotic animals and farm animals, including mammals, rodents, and the like. More preferred animals include horses, dogs, and cats.

The immunogenicity of DKK1 peptides have been examined using peptide immunization in transgenic mice, and generated and characterized DKK1 peptide-specific CTLs from healthy blood donors and cancer patients. As discussed below in detail in the Examples, to determine DKK1 as a TAA in MM, two of the DKK1 peptides P20 and P66 were synthesized after searching DKK1 sequence for HLA-A*0201 binding motifs and analyzed. To enhance binding affinity, heteroclitic peptides for P20 (Py20) and P66 (P66v) were synthesized. Tourdot, S., et al., A General Strategy to Enhance Immunogenicity of Low Affinity HLA-A2.1-Associated Peptides: Implication in the Identification of Cryptic Tumor Epitopes, Eur J. Immunol. 2000, 30:3411-3421; Chen, J. L., et al., Identification of NY-ESO-1 Peptide Analogues Capable of Improved Stimulation of Tumor-Reactive CTL, J Immunol, 2000, 165:948-955. As P20 and Py20 had similar binding affinity while P66v had much higher binding affinity than P66, P20 (SEQ. ID NO. 1) and P66v (SEQ. ID. NO. 4) was used in the experiments. These peptides were immunogenic in vivo. After a single immunization of HLA-A*0201 transgenic mice with the peptides, splenocytes from immunized mice contained detectable peptide-specific T cells, analyzed as peptide-tetramer⁺ and IFN-γ-secreting CD8⁺ T cells that were able to kill target cells. By DKK1 peptide-tetramer staining, naturally occurring DKK1-specific CD8⁺ T cells in the PBMCs of myeloma patients were detected, showing that DKK1 is a shared TAA among different patients. In focusing on the DKK1 peptides presented by HLA-A*0201 molecules, DKK1 peptide-specific CTLs were found to recognize and lyse autologous and allogeneic DKK1⁺/HLA-A*0201⁺, but not DKK1⁻/HLA-A*0201⁺ or DKK1⁺/HLA-A*0201⁻ myeloma cells. Furthermore, the findings prove that these DKK1 peptides are also naturally processed and presented by myeloma cells in the context of surface MHC class I molecules.

Further, these DKK1 peptide-specific CTLs have strong cytolytic activity against DKK1 peptide-pulsed T2 cells and autologous dendritic cells, DKK1⁺/HLA-A*0201⁺ HMCLs U266 and IM-9. More importantly, while these DKK1 peptide-specific CTLs had strong cytolytic activity against primary myeloma cells from HLA-A*0201⁺, they were not cytolytic against HLA-A*0201⁻ patients.

Generally, the demonstration of autologous idiotype-specific T cells and evidence of clinical response to allogeneic donor lymphocyte infusions show that anti-myeloma responses can be generated. Wen, Y. J., et al., Idiotype-Specific Cytotoxic T Lymphocytes in Multiple Myeloma: Evidence For Their Capacity to Lyse Autologous Primary Tumor Cells, Blood. 2001, 97:1750-1755; Tricot, G., et al., Graft-Versus-Myeloma Effect: Proof of Principle, Blood. 1996, 87:1196-1198; Orsini, E., et al., Expansion of Tumor-Specific CD8+ T Cell Clones in Patients With Relapsed Myeloma After Donor Lymphocyte Infusion, Cancer Res. 2003, 63:2561-2568; Salama, M., et al., Donor Leukocyte Infusions for Multiple Myeloma, Bone Marrow Transplant. 2000, 26:1179-1184; Lokhorst, H. M., et al., Donor Leukocyte Infusions are Effective in Relapsed Multiple Myeloma After Allogeneic Bone Marrow Transplantation, Blood. 1997, 90:4206-4211. Specific cytotoxic T lymphocyte (CTL)-mediated immunotherapy for MM can be achieved by vaccination using the idiotype proteins isolated from the serum of patients. Stevenson, F. K., et al., Preparing the Ground for Vaccination Against Multiple Myeloma, Immunol Today. 2000, 21:170-171. However, the idiotype proteins represent a unique myeloma-associated antigen and thus cannot provide shared immunotherapy for various patients with MM. Immunotherapy in a combination with high-dose chemotherapy holds great promise for the treatment of MM and other cancers.

Also, in addition to myeloma cells, DKK1 mRNA has been detected in some normal tissues such as testis, prostate, placenta, and uterus. Whether DKK1 protein is also expressed in these tissues remained to be examined. Based on the expression pattern, however, DKK1 resembles cancer-testis antigens, because the most commonly used cancer-testis antigens NY-ESO-1 and MAGE are also found in, in addition to tumors and testis, the uterus, placenta, ovary, and even brain. Scanlan, M. J., et al., Cancer/Testis Antigens: an Expanding Family of Targets for Cancer Immunotherapy, Immunol Rev. 2002, 188:22-32; Sacha Gnjatic, H. N., et al., NY-ESO-1: Review of an Immunogenic Tumor Antigen, Elsevier Inc, 2006.

DKK1 peptide-specific CTLs have been tested against normal blood cells, including dendritic cells, B cells, and PBMCs (peripheral blood mononuclear cells), which do not express DKK1. As expected, the CTLs did not kill these cells but lysed MSCs (mesenchymal stem cells) because MSCs expressed DKK1 protein as MSCs display immunosuppressive activity in both animals and humans. Uccelli, A., et al., Immunoregulatory Function of Mesenchymal Stem Cells, Eur J. Immunol. 2006, 36:2566-2573. Furthermore, there is evidence indicating that chemotherapy drugs such as thalidomide and lenalidomide upregulate DKK1 mRNA expression in myeloma cells and dexamethasone enhanced DKK1 expression in osteoblasts. Colla, S., et al., The Oxidative Stress Response Regulates DKK1 Expression Through the JNK Signaling Cascade in Multiple Myeloma Plasma Cells, Blood. 2007; Ohnaka, K., et al., Glucocorticoid Enhances the Expression of Dickkopf-1 in Human Osteoblasts: Novel Mechanism of Glucocorticoid-Induced Osteoporosism, Biochem Biophys Res Commun. 2004, 318:259-264. Taken together, the reactivity and impact of DKK1 peptide-specific CTLs on normal tissues that express DKK1 protein must be determined, and whether chemotherapy could further enhance the sensitivity of myeloma cells and MSCs to the CTL-mediated cytolysis.

Generally, CTL recognition of target cells via their T-cell receptor activates two distinct mechanisms of cell lysis. Kagi, D., et al., Fas and Perforin Pathways as Major Mechanisms of T Cell-Mediated Cytotoxicity, Science. 1994, 265:528-530; Kojima, H., et al., Two Distinct Pathways of Specific Killing Revealed by Perforin Mutant Cytotoxic T lymphocytes, Immunity. 1994, 1:357-364. The first is granule exocytosis mediated by the pore-forming perforin and granzyme A and B. The second involves interaction between the FasL on effector cells and Fas molecules expressed on the target cells.

The DKK1 peptide-specific CTLs lyse the target cells mainly via the perforin-mediated pathway, since the cells expressed high levels of granzyme B and perforin but not FasL. These findings are of special importance in view of published results on Fas expression on myeloma cells. A previous study showed that Fas antigen point mutation was detected in 10% of patients' bone marrow samples. The mutations were located in the cytoplasmic region that is involved in transduction of an apoptotic signal and, thus, render the cells resistant to Fas-induced apoptosis. Landowski, T. H., et al., Mutations in the Fas Antigen in Patients With Multiple Myeloma, Blood. 1997, 90:4266-4270. Furthermore myeloma cells induced to be drug resistant also became resistant to Fas-mediated apoptosis. Landowski, T. H., et al., Selection for Drug Resistance Results in Resistance to Fas-Mediated Apoptosis. Blood. 1997, 89:1854-1861. Thus, the use of CTLs that are cytotoxic via the Fas-mediated pathway may be limited, but the pore-forming CTLs can be used for the treatment of drug-resistant myeloma.

As described in the Examples, DKK1 peptides for HLA-A*0201 have been shown to be immunogenic by in vivo immunization in HLA-A*0201 transgenic mice. Using peptide-tetramers, low frequencies of DKK1 peptide-specific CD8⁺ T cells in myeloma patients and generated peptide-specific T-cell lines and clones from HLA-A*0201⁺ blood donors and myeloma patients. The T cells efficiently lysed peptide-pulsed but not unpulsed T2 or autologous dendritic cells, DKK1⁺/HLA-A*0201⁺ myeloma cell lines U266 and IM-9, and HLA-A*0201⁺ primary myeloma cells from patients. No killing was observed on DKK1⁺/HLA-A*0201⁻ myeloma cell lines and primary myeloma cells or HLA-A*0201⁺ normal lymphocytes including B cells. Hence, the T cells were shown to be potent cytotoxic T cells as they recognized DKK1 peptides naturally presented by myeloma cells in the context of HLA-A*0201 molecules.

The following examples are provided to more fully illustrate some of the embodiments of the present disclosure. It should be appreciated by those of skill in the art that the techniques disclosed in the examples which follow represent techniques discovered by the inventors to function well in the practice of the invention, and thus can be considered to constitute exemplary modes for its practice. However, those of skill in the art should, in light of the present disclosure, appreciate that many changes can be made in the specific embodiments that are disclosed and still obtain a like or similar result without departing from the spirit and scope of the disclosure.

### EXAMPLE 1

Human myeloma cell lines (HMCLs) used include U266, IM-9, XG1, ARP-1, ARK, J41MT, MM1-144, AKMM1, RPMI-8226, MM.1S, and MM.1R. All cell lines were maintained in RPMI-1640 medium (Fisher Scientific, Herndon, Va.) supplemented with 10% fetal bovine serum (Atlanta Biologicals, Lawrenceville, Ga.). K562 (American Type Culture Collection; Rockville, Md.) were used as natural killer (NK) cell-sensitive targets. Primary myeloma cells were isolated from myeloma patients' bone marrow aspirates by density centrifugation and anti-human CD138 antibody-coated magnetic microbeads (Miltenyi Biotec, Bergisch Gladbach, Germany). Qian, J., et al., Targeting Heat Shock Proteins for Immunotherapy in Multiple Myeloma: Generation of Myeloma-Specific CTLs Using Dendritic Cells Pulsed With Tumor-Derived GP96, Clin Cancer Res. 2005, 11:8808-8815. Aliquots of purified myeloma cells were cryopreserved in liquid nitrogen until use.

### Generation of Dendritic Cells (DCs)

Monocyte-derived mature DCs were generated from peripheral blood mononuclear cells (PBMCs) using standard protocol. Qian, J., et al., Targeting Heat Shock Proteins for Immunotherapy in Multiple Myeloma: Generation of Myeloma-Specific CTLs Using Dendritic Cells Pulsed With Tumor-Derived GP96, Clin Cancer Res. 2005, 11:8808-8815; Romani, N., et al., Generation of Mature Dendritic Cells from Human Blood. An Improved Method with Special Regard to Clinical Applicability, J Immunol Methods. 1996, 196:137-151; Sallusto, F., et al., Efficient Presentation of Soluble Antigen by Cultured Human Dendritic Cells is Maintained by Granulocyte/Macrophage Colony-Stimulating Factor Plus Interleukin 4 and Downregulated by Tumor Necrosis Factor Alpha, J Exp Med. 1994, 179:1109-1118; Anton, D., et al., Generation of Dendritic Cells from Peripheral Blood Adherent Cells in Medium with Human Serum, Scand J. Immunol. 1998, 47:116-121. Briefly, PBMCs were allowed to adhere in culture flasks for 2 hours and non-adherent cells were collected and cryopreserved for future use. The adherent cells were cultured in Aim-V medium (Invitrogen Co., Grand Island, N.Y.) supplemented with GM-CSF (10 ng/mL) and IL-4 (10 ng/mL, both from R&D Systems, Minneapolis, Minn.), with further addition of cytokines every other days. After 5 days of culture, DCs were induced to maturation by addition of TNF-α (10 ng/mL) and IL-113 (10 ng/mL, both from R&D Systems) for 48 hours.

### Immunophenotyping and Intracellular Cytokine Staining

Phycoerythrin (PE) or fluorescein isothiocyanate (FITC) conjugated monoclonal antibodies (mAbs) were added to cell pellets, incubated for 30 minutes on ice, and washed 3 times before analysis. Intracellular cytokine staining was performed using the Cytofix/Cytoperm kit (BD PharMingen, San Diego, Calif.) according to the manufacturer's recommendations. Samples were analyzed using a flow cytometer (FACSCaliber; Becton Dickinson, Mountain View, Calif.).

### Peptide-T2 Cell Binding Assay

All peptides were synthesized in the Peptide Synthesis Facility at M. D. Anderson Cancer Center. Purity of synthetic peptides was confirmed to be >98% by reverse-phase high-performance liquid chromatography and mass spectrometry. Synthetic peptides were dissolved in dimethylsulfoxide (DMSO; Sigma, St. Louis, Mo.), and stored at −20° C. until use. Peptides binding to HLA-A*0201 molecules were measured using the T2 cell line according to a protocol previously described. Nijman, H. W., et al., Identification of Peptide Sequences that Potentially Trigger HLA-A2.1-Restricted Cytotoxic T Lymphocytes, Eur J. Immunol. 1993, 23:1215-1219. Briefly, T2 cells were incubated overnight with 3 μg/mL of β₂-microglobulin (Sigma) and different concentrations of peptides, followed by wash and incubation with FITC-labeled anti-HLA-A*0201 mAb BB7.2 (BD PharMingen). After washing, cells were analyzed for the levels of HLA-A*0201 expression by flow cytometry. HLA-A*0201 expression was quantified according to the formula [(mean fluorescence with peptide−mean fluorescence without peptide)/mean fluorescence without peptide]×100.

### Determination of In Vivo Immunogenicity of the Peptides

HLA-A*0201 transgenic (Tg[HLA-A2.1]) mice were purchased from Jackson Laboratory (Bar Harbor, Me.). Ishioka, G. Y., et al., Utilization of MHC Class I Transgenic Mice for Development of Minigene DNA Vaccines Encoding Multiple HLA-Restricted CTL Epitopes, J. Immunol. 1999, 162:3915-3925; Alexander, J., et al., A Decaepitope Polypeptide Primes for Multiple CD8+ IFN-Gamma and Th Lymphocyte Responses: Evaluation of Multiepitope Polypeptides as a Mode for Vaccine Delivery, J. Immunol. 2002, 168:6189-6198; Tangri, S., et al., Structural Features of Peptide Analogs of Human Histocompatibility Leukocyte Antigen Class I Epitopes that are More Potent and Immunogenic Than Wild-Type Peptide, J Exp Med. 2001, 194:833-846. Mice were maintained at the animal facility and studies were approved by the Institutional Animal Care and Use Committees of The University of Texas M. D. Anderson Cancer Center.

For immunization, peptides were diluted in PBS at room temperature, mixed, and emulsified with an equal volume of incomplete Freund's adjuvant (Sigma). Groups of three mice were immunized subcutaneously (s.c.) at the tail base with 100 μL of peptide emulsion containing 100 μg peptides. Two weeks following the immunization, mice were sacrificed and splenocytes were isolated for in vitro studies.

### Reverse Transcriptase—Polymerase Chain Reaction (RT-PCR) for Detecting DKK1 mRNA Expression

RT-PCR was performed using a PTC-1000™ programmable thermal controller (MJ Research, INC) with QuantiTect SYBR Green PCR kit (Qiagen, Valencia, Calif.) according to the manufacturer's instructions. Total RNA isolated from tumor cells with an RNeasy Mini kit (Qiagen), and human total RNA master multi-tissue panel from Clontech Laboratory (Mountain View, Calif.) were used. Random-primed cDNA synthesis was performed using QuantiTect Reverse Transcription Kit (Qiagen). For amplifications, the following primers were designed: DKK1 forward, 5′-AGA CCA TTG ACA ACT ACC AGC CGT-3′; reverse, TCT GGA ATA CCC ATC CAA GGT GCT-3′; and GAPDH forward, 5′-CCT CCG GGA AAC TGT GGC GTG ATG G-3′; reverse, 5′-AGA CGG CAG GTC AGG TCC ACC ACT G-3′. Each of the primer sets was confirmed by running samples on agarose gels. GAPDH transcript levels were used to normalize the amount of cDNA in each sample.

### Western Blot Analysis

Western blot analysis was employed to detect DKK1 protein expression in myeloma cells. Cell lysates were prepared from purified primary myeloma cells and cell lines and subjected to sodium dodecyl sulfate-polyacrylamide gel electrophoresis (SDS-PAGE) analysis. After transfer to nitrocellulose membrane and subsequent blocking, the membranes were immunoblotted with goat anti-human DKK1 antibody (R&D System) and visualized with HRP-conjugated donkey anti-goat IgG antibody (Santa Cruz Biotechnology, Santa Cruz, Calif.), followed by an enhanced chemiluminescence (Pierce Biotechnology, Rockford, Ill.) and autoradiography.

### Generation of DKK1-Specific T-Cell Lines and Clones

DKK1-specific T cells were generated from PBMCs of HLA-A*0201⁺ blood donors and myeloma patients by repeated stimulation of autologous T cells with DKK1 peptide-loaded mature DCs. Briefly, the non-adherent cells of PBMCs (2×10⁶/mL; used as T-cell population) were cocultured in 50 mL tissue-culture flasks at 37° C. in 5% CO₂ for 7-10 days in Aim-V medium supplemented with 10% pooled human serum (T-cell medium) with mature DCs (2×10⁵/mL) preincubated with DKK1 peptides at a final concentration of 50 μg/mL at 37° C. for 2 h. After culture, T cells were collected and restimulated with DKK1 peptide-pulsed autologous mature DCs every week, and the cultures were fed every 5 days with fresh medium containing recombinant IL-2 (20 IU/mL), IL-7 (5 ng/mL) and IL-15 (5 ng/mL; all from R&D System). Induction of DKK1-specific T cells was monitored weekly using T-cell proliferation assay and DKK1 peptide-HLA-A*0201 tetramers (synthesized by MHC Tetramer Laboratory, Baylor College of Medicine, Houston, Tex.). After 3-4 cycles of in vitro stimulation and selection, T-cell lines were established, and T-cell clones were obtained by limiting-dilution assay. T-cell lines and clones were expanded in T-cell medium containing IL-2 (100 IU/mL), IL-7 (5 ng/mL), and IL-15 (5 ng/mL) for 2 weeks and subjected to functional tests.

### Proliferation Assays

T cells (5×10⁴/100 μL/well) were seeded into 96-well U-bottom tissue culture plates (Corning Incorporated, Corning, N.Y.) in T-cell medium. Varying numbers of autologous mature DCs loaded with or without DKK1 peptides were added to the plates and cultured for 4 days at 37° C. in 5% CO₂. T-cell proliferation was measured after overnight incubation with ³[H]-thymidine (0.5 μCi/0.037 MBq/well). Results are expressed as mean count per minute (CPM) of triplicate cultures.

In some experiments, cultured T cells were labeled with 5(6)-carboxyfluorescein diacetate succinimidyl ester (CFSE, 5 μM; Molecular Probes, Eugene, Oreg.) for 10 minutes at 37° C. After washing, labeled T cells were seeded into 96-well U-bottom plates and incubated with various numbers of stimulatory cells for 7 days. Flow cytometry analysis was used to detect dilution of CFSE. Fulcher, D., et al., Carboxyfluorescein Succinimidyl Ester-Based Proliferative Assays for Assessment of T Cell Function in the Diagnostic Laboratory, Immunol Cell Biol. 1999, 77:559-564.

### Cytotoxicity Assay

The standard 4-h ⁵¹Cr-release assay was performed to measure cytolytic activity of the T cells with target cells including autologous DCs or T2 cells loaded with or without peptides, myeloma cell lines and primary myeloma cells isolated from patients. Qian, J., et al., Targeting Heat Shock Proteins for Immunotherapy in Multiple Myeloma: Generation of Myeloma-Specific CTLs Using Dendritic Cells Pulsed With Tumor-Derived GP96, Clin Cancer Res. 2005, 11:8808-8815. Target cells were incubated with 100 μCi of ⁵¹Cr-sodium chromate for 1 h, washed extensively, seeded (1×10⁴ cells/well) into 96-well U-bottom plates in T-cell medium, and cocultured for 4 h with various numbers of T cells. All assays were performed in triplicates. Results are expressed as mean percentage of ⁵¹Cr release calculated as follows: [(sample counts−spontaneous counts)/(maximum counts−spontaneous counts)]×100%. Spontaneous release was less than 20% of the maximum ⁵¹Cr uptake.

To determine whether the cytolytic activity was restricted by major histocompatibility complex (MHC) class I or II molecules, 20 μg/mL mAbs against HLA-ABC (W6/32) or HLA-A*0201 (BB7.2; both purchased from Serotec Ltd., Oxford, UK), HLA-DR (B8.12.2; Immunotech, Marseilles, France), or isotypic controls (Immunotech) were added to the cultures at the start of the assay.

### Enzyme-Linked Immunosorbent Assays (ELISA)

ELISA for IFN-γ was used to measure the secreted cytokine. Supernatants of T cells cocultured with or without antigen presenting cells (APCs) were collected on day 3, and the amounts of secreted IFN-γ in the supernatants were quantified using a commercially available ELISA kit (R&D Systems).

### ELISPOT Assay

Detailed methods of the ELISPOT assay for enumeration of antigen-specific, IFN-γ-secreting cells have been described previously. Yi, Q., et al., T-Cell Stimulation Induced by Idiotypes on Monoclonal Immunoglobulins in Patients With Monoclonal Gammopathies, Scand J. Immunol. 1993, 38:529-534; Yi, Q., et al., Idiotype Reactive T-Cell Subsets and Tumor Load in Monoclonal Gammopathies, Blood. 1995, 86:3043-3049. The number of IFN-γ spots was enumerated by an automate ImmunoSpot analyzer (Cellular Technology Ltd, Cleveland, Ohio). All samples were run in duplicates. Data are expressed as the mean number of IFN-γ-secreting cells/10⁴ T cells.

### Statistical Analysis

The Student t test was used to compare various experimental groups. A P value less than 0.05 was considered statistically significant. Unless otherwise indicated, means and standard deviation (SD) are shown.

### Results

### DKK1 is Widely Expressed in Myeloma But Not Normal Cells

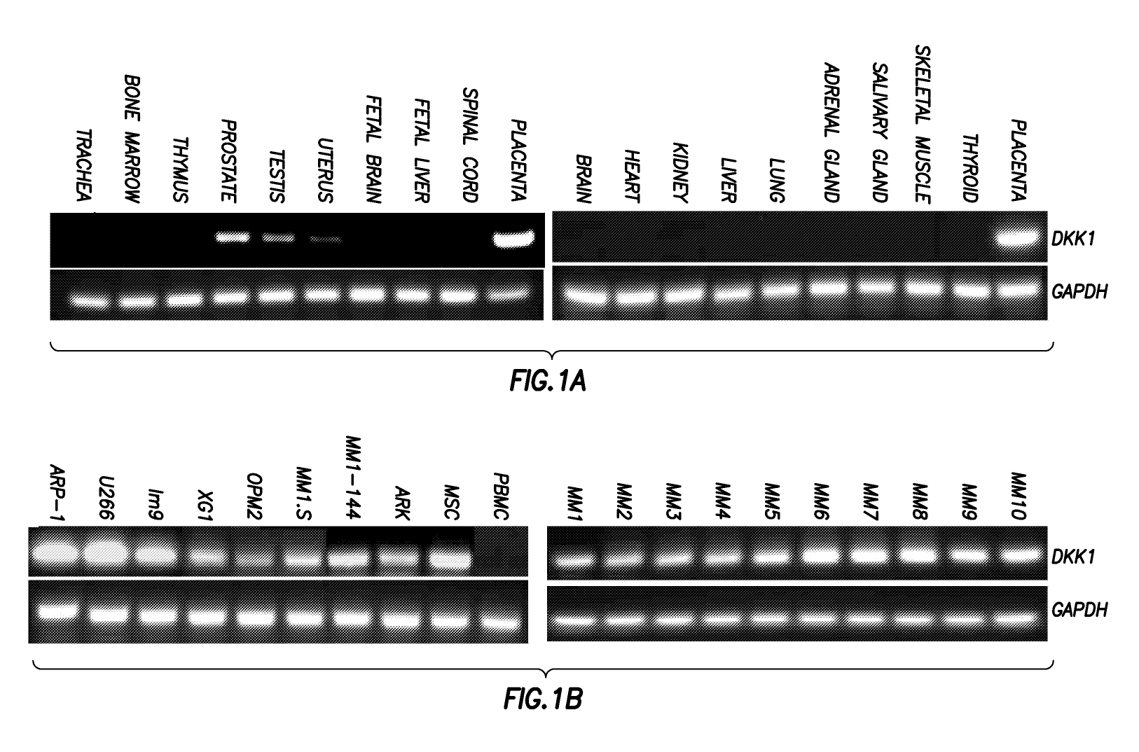

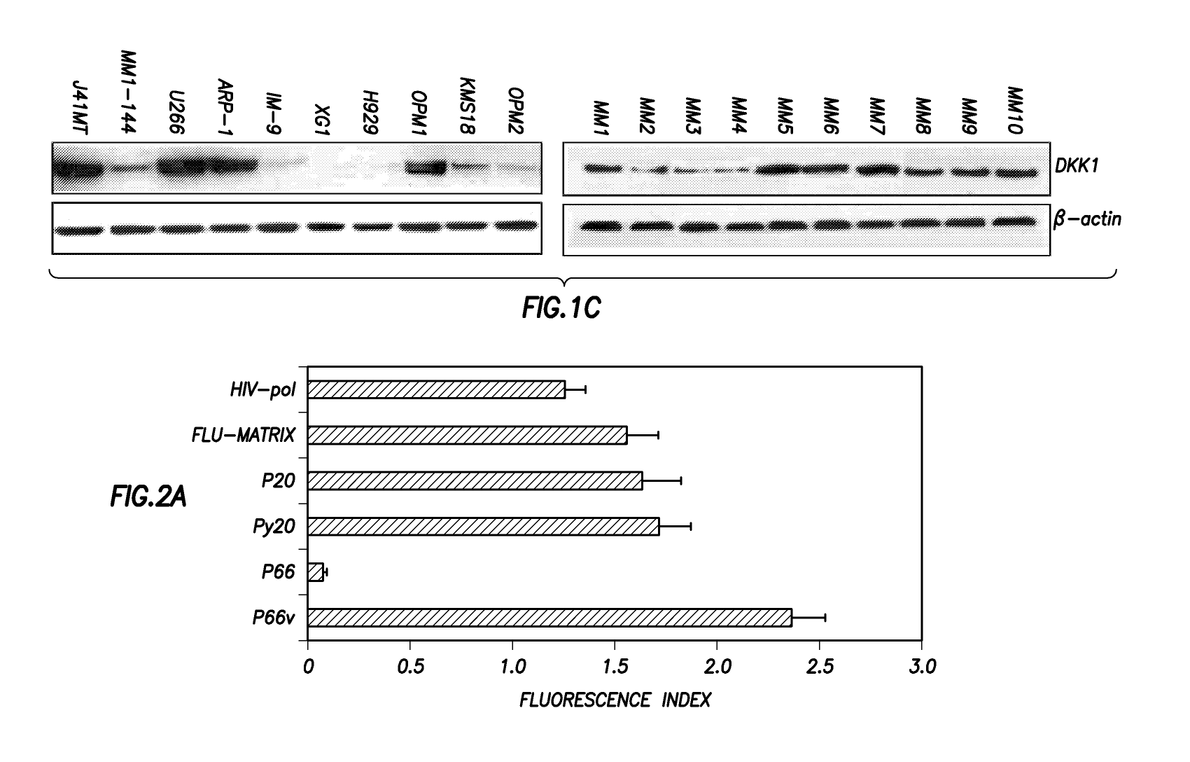

RT-PCR and Western blot analyses were used to examine the expression of DKK1 in normal human tissues and myeloma cells. As shown in FIG. 1A, DKK1 mRNA was not detected in most human tissues except prostate, testis, placenta, and uterus. DKK1 mRNA was detected in all 8 myeloma cell lines and primary myeloma cells from 10 patients examined, and in MSCs but not PBMCs from a healthy blood donor. (FIG. 1B). Similarly, DKK1 protein was detected in 7 out of 10 myeloma cells lines and all primary myeloma cells from 10 mM patients (FIG. 1C).

### Selection of DKK1 Peptides

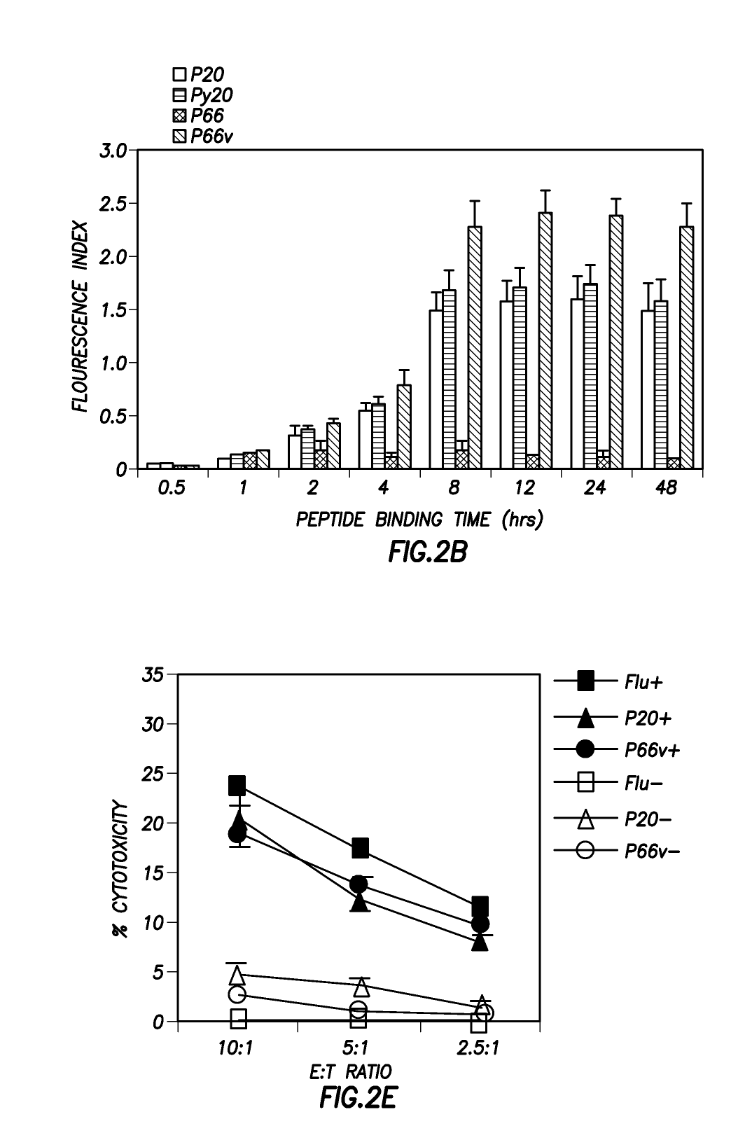

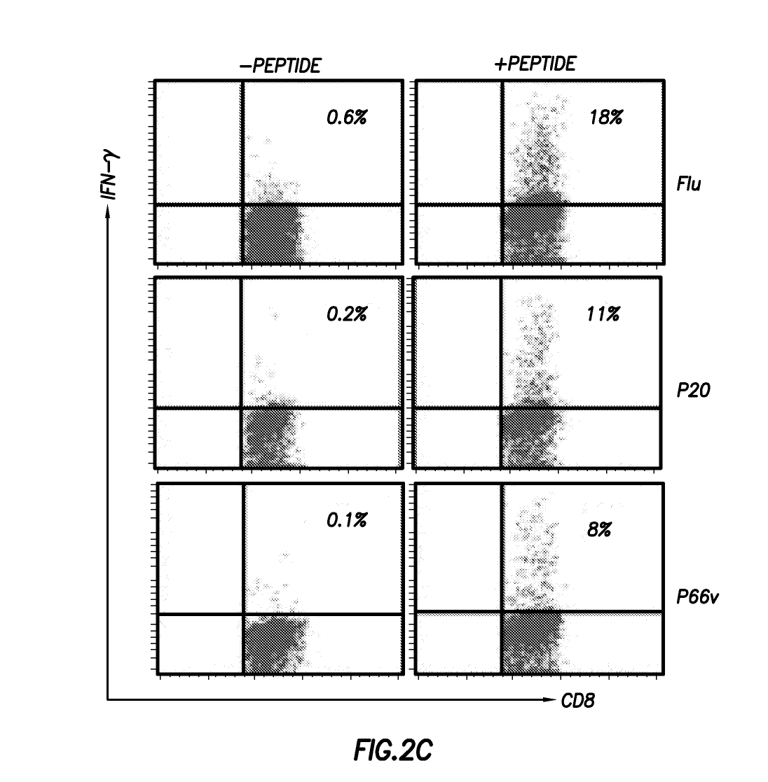

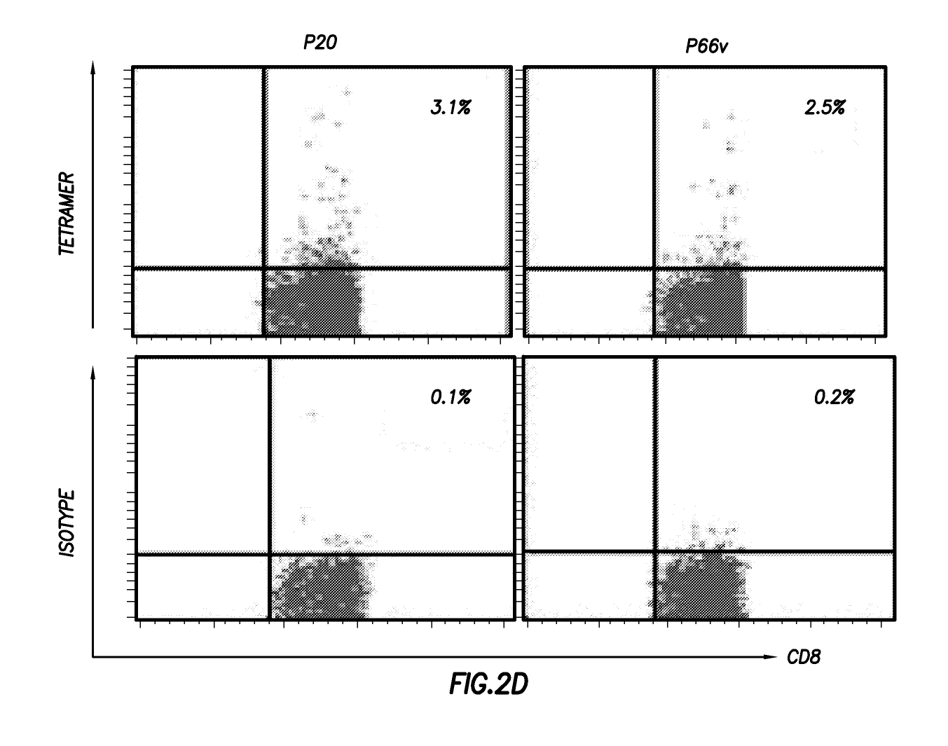

The sequence of DKK1 was reviewed for peptides that could potentially bind to HLA-A*0201 using peptide binding database (http://www.bimas.dcrt.nih.gov). After comparing the predictive binding scores, we identified and selected two peptides that could potentially bind with HLA-A*0201 molecules; one with the highest binding score, P20 (SEQ ID NO 1) and one with a low score, P66. As shown in Table 1, heteroclitic peptides, by replacing position-1 amino acid with tyrosine (Py20) or c-terminal amino acid with valine (P66v), were made, which had significantly higher predictive binding scores. Tourdot, S., et al., A General Strategy to Enhance Immunogenicity of Low Affinity HLA-A2.1-Associated Peptides: Implication in the Identification of Cryptic Tumor Epitopes, Eur J. Immunol. 2000, 30:3411-3421; Chen, J. L., et al., Identification of NY-ESO-1 Peptide Analogues Capable of Improved Stimulation of Tumor-Reactive CTL, J Immunol, 2000, 165:948-955. Based on the binding affinity (FIG. 2A) and stability (FIG. 2B) measured by peptide-T2 binding assay, we selected the unmodified P20 and heteroclitic P66v for the following experiments. Peptides from influenza virus matrix protein (Flu-matrix), and HIV type 1 reverse transcriptase (HIV-pol) were used as controls. Dyall, J., et al., Lentivirus-Transduced Human Monocyte-Derived Dendritic Cells Efficiently Stimulate Antigen-Specific Cytotoxic T Lymphocytes, Blood. 2001, 97:114-121; Tsomides, T. J., et al., An Optimal Viral Peptide Recognized by CD8+ T Cells Binds Very Tightly to the Restricting Class I Major Histocompatibility Complex Protein on Intact Cells But Not to the Purified Class I Protein, Proc Natl Acad Sci USA. 1991, 88:11276-11280.

### In Vivo Immunogenicity of Synthetic DKK1 Peptides

To examine whether the peptides were able to immunize HLA-A*0201 transgenic mice, we injected 100 μg per mouse of peptides P20 (SEQ. ID NO. 1) and P66v (SEQ. ID. NO. 4), and Flu-matrix peptide as positive control s.c. to the mice (3 mice per peptide) according to established protocol. Ishioka, G. Y., et al., Utilization of MHC Class I Transgenic Mice for Development of Minigene DNA Vaccines Encoding Multiple HLA-Restricted CTL Epitopes, J. Immunol. 1999, 162:3915-3925; Alexander, J., et al., A Decaepitope Polypeptide Primes for Multiple CD8+ IFN-Gamma and Th Lymphocyte Responses: Evaluation of Multiepitope Polypeptides as a Mode for Vaccine Delivery, J. Immunol. 2002, 168:6189-6198; Tangri, S., et al., Structural Features of Peptide Analogs of Human Histocompatibility Leukocyte Antigen Class I Epitopes that are More Potent and Immunogenic Than Wild-Type Peptide, J Exp Med. 2001, 194:833-846. Two weeks after the immunization, mice were sacrificed, and splenocytes were collected, restimulated with the immunizing peptides for 5 days, and subjected to analyses. In vivo immunization successfully generated peptide-specific T cells, detected as specific IFN-γ-expressing (FIG. 2C) and peptide (P20 or P66v)-HLA-A*0201 tetramer⁺ CD8⁺ T cells (FIG. 2D). Furthermore, the splenocytes displayed strong cytolytic activity against peptide-pulsed, but not unpulsed, murine DCs (FIG. 2E). It appeared that P20 (SEQ. ID NO. 1) and P66v (SEQ. ID. NO. 4) were as immunogenic as the Flu-matrix peptide in immunizing the mice. These results indicate that the DKK1 peptides were able to induce a strong peptide-specific CTL response in HLA-A*0201 transgenic mice.

### Generation of DKK1 Peptide-Specific T-Cell Lines

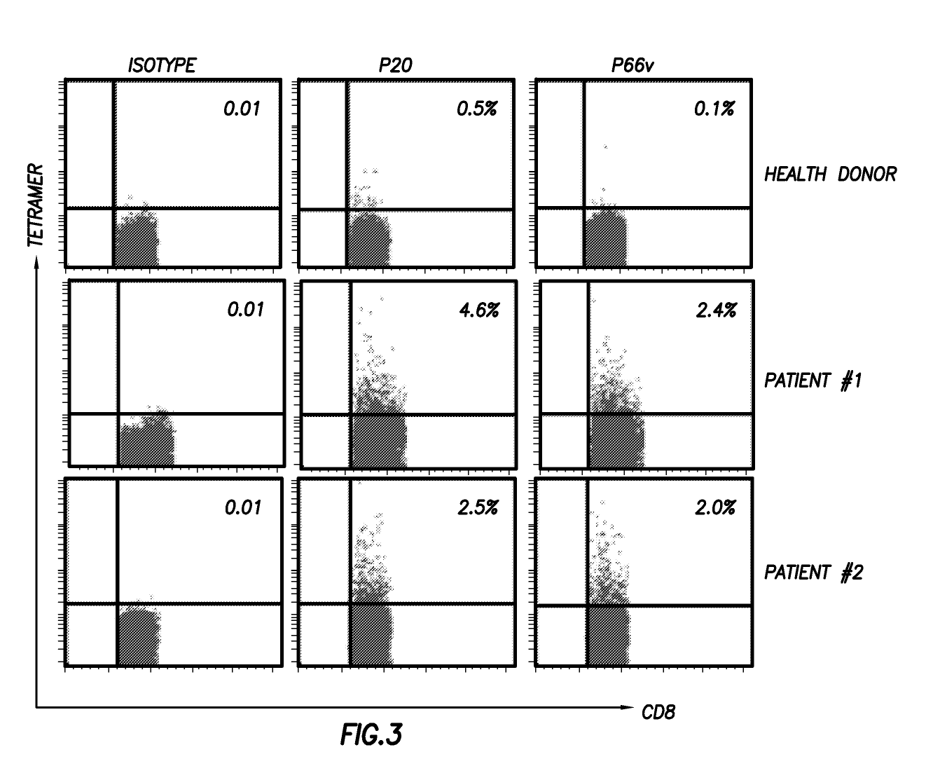

We first examined whether DKK1 peptide-specific CTL precursor cells are present in patients with MM. By using peptide (P20 [SEQ. ID. NO. 1] or P66v [SEQ. ID. NO. 4])-HLA-A*0201 tetramers to stain T cells of myeloma patients and healthy blood donors, we show that DKK1 peptide-specific CD8⁺ T cells, although at low frequencies, were detected in two myeloma patients examined, whereas the frequency of such T cells was much lower in the healthy blood donor (FIG. 3).

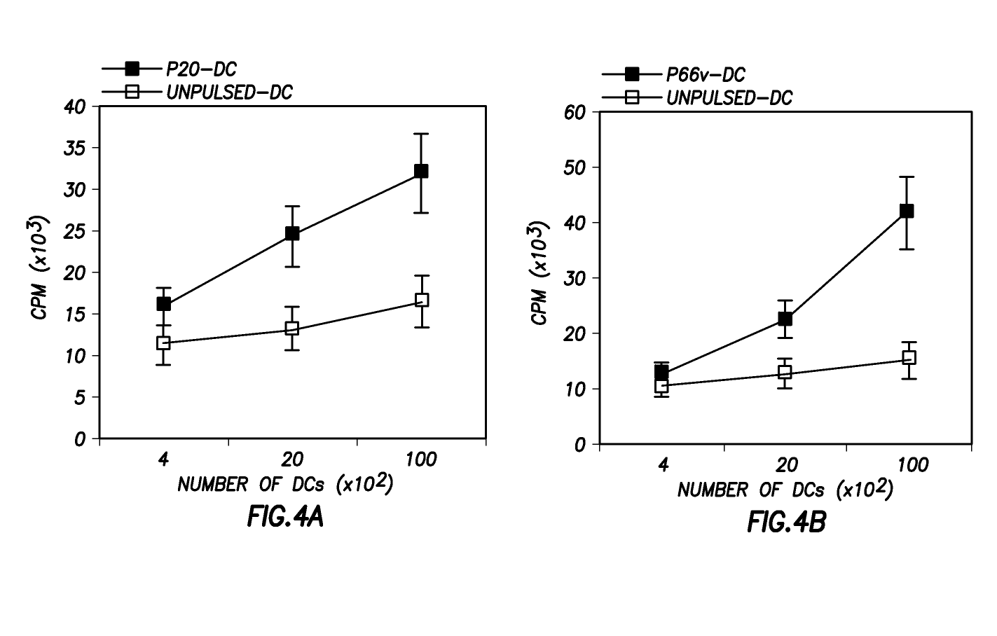

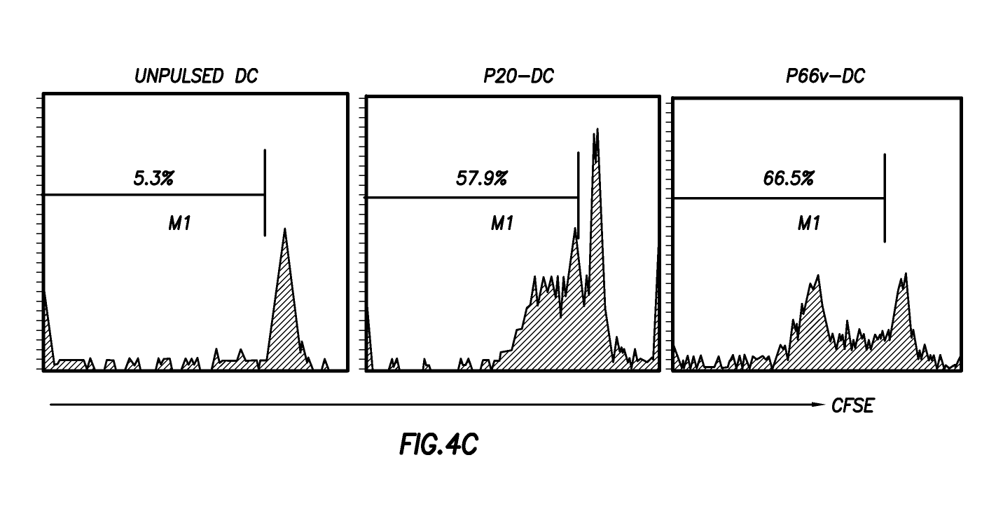

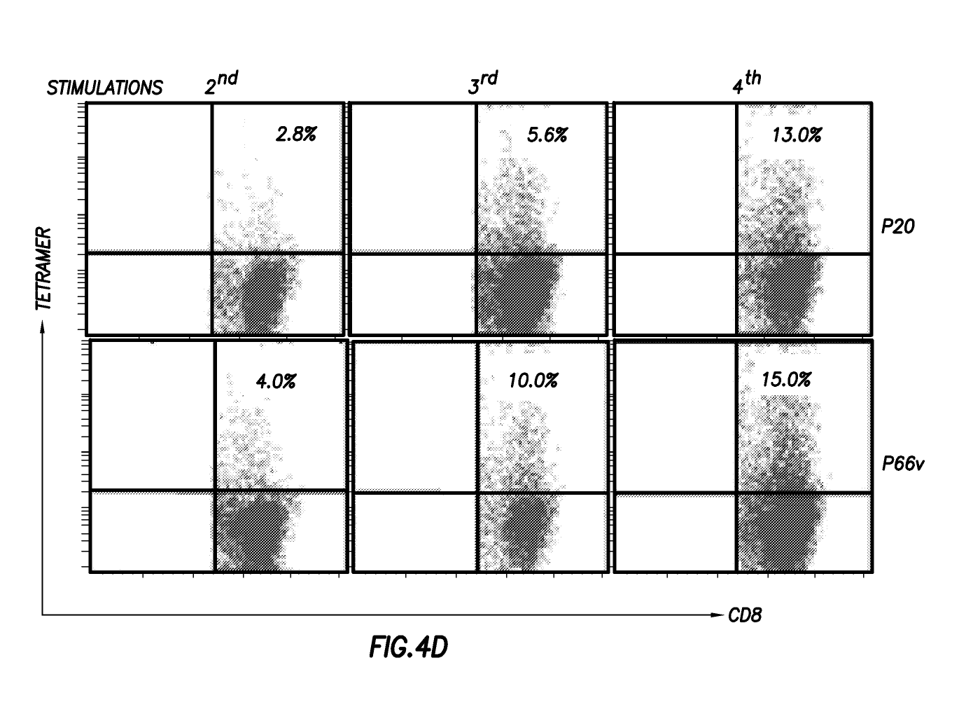

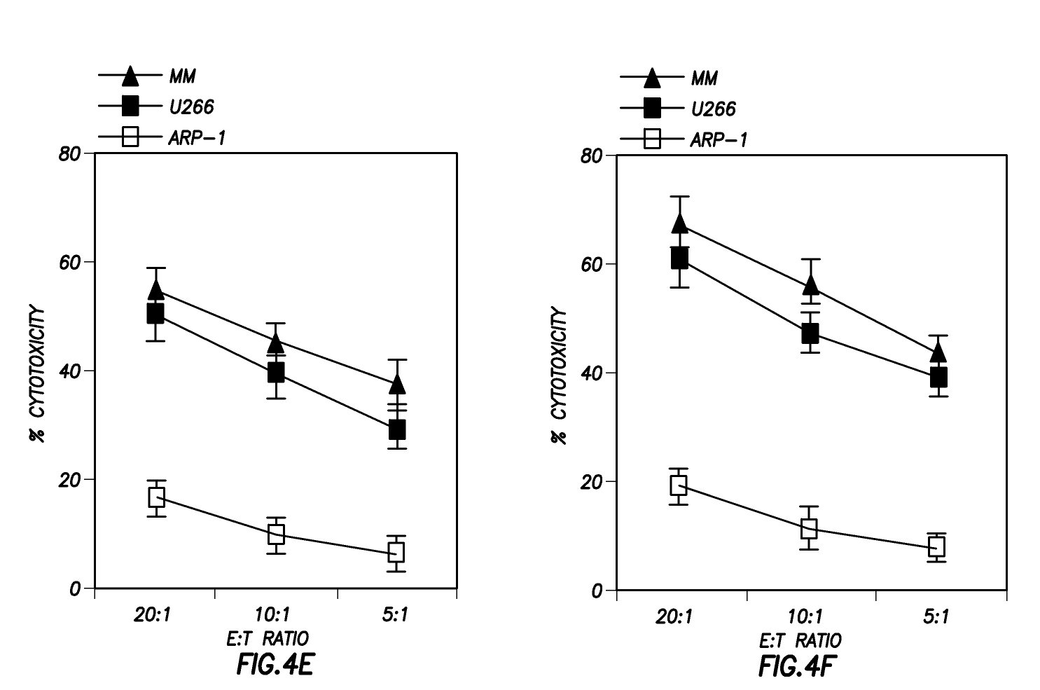

To generate DKK1 peptide-specific T cells from HLA-A*0201⁺ blood donors and myeloma patients, autologous mature DCs pulsed with peptides were used as APCs. After 3 to 4 rounds of in vitro stimulation, T-cell lines were obtained, which proliferated in response to autologous DCs pulsed, but not unpulsed, with DKK1 peptides P20 (P<0.01, compared with unpulsed DC; FIG. 4A) or P66v (P<0.01, compared with unpulsed DC; FIG. 4B). The same results were also obtained with a CFSE-labeling assay to measure T-cell proliferation (FIG. 4C). Using peptide-tetramer staining, we show that the frequencies of peptide-specific CD8⁺ T cells increased during in vitro stimulation; from 3 to 4% of specific T cells at the second stimulation to 13 to 15% at fourth stimulation (FIG. 4D). The standard 4-h ⁵¹Cr-release assay was used to examine the cytotoxicity of the T cells. As exemplified by the results obtained with a T-cell line specific for P20 (FIG. 4E) or P66v (FIG. 4F) generated from myeloma patients, the T cells specifically lysed autologous primary myeloma cells and DKK1⁺/HLA-A*0201⁺ U266 but not DKK1⁺/HLA-A*0201⁻ ARP-1 cells. These results suggest that the T-cell lines recognized the DKK1 peptides that are naturally processed and presented in the context of HLA-A*0201 molecules on myeloma cells.

### Cloning and Characterizing DKK1 Peptide-Specific CTLs

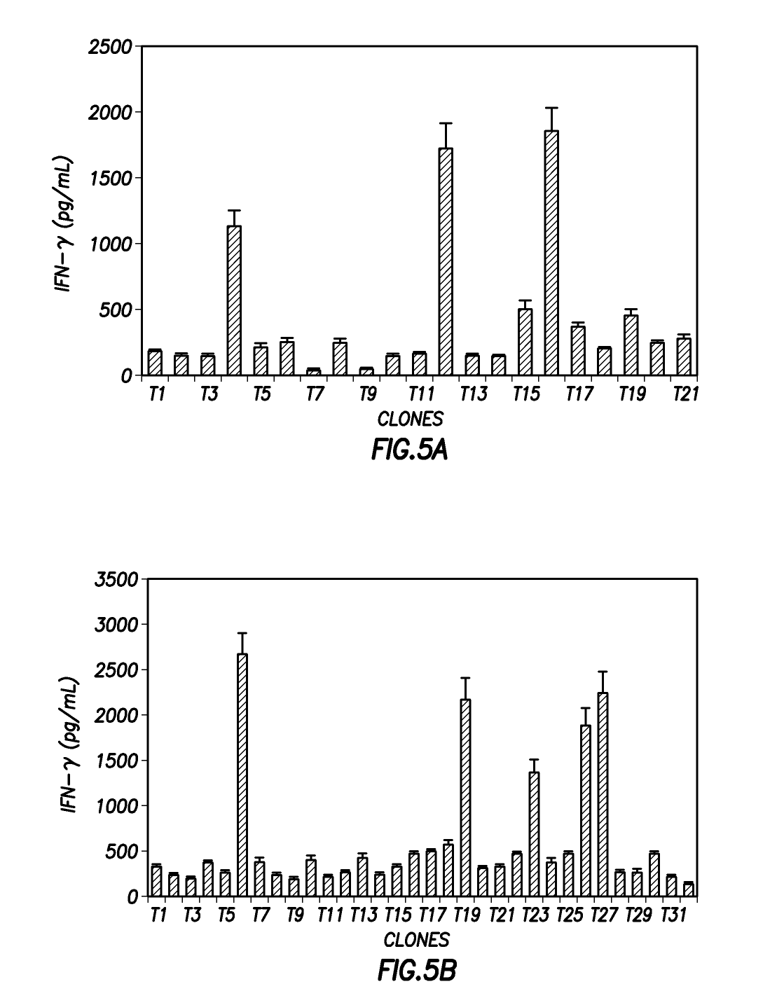

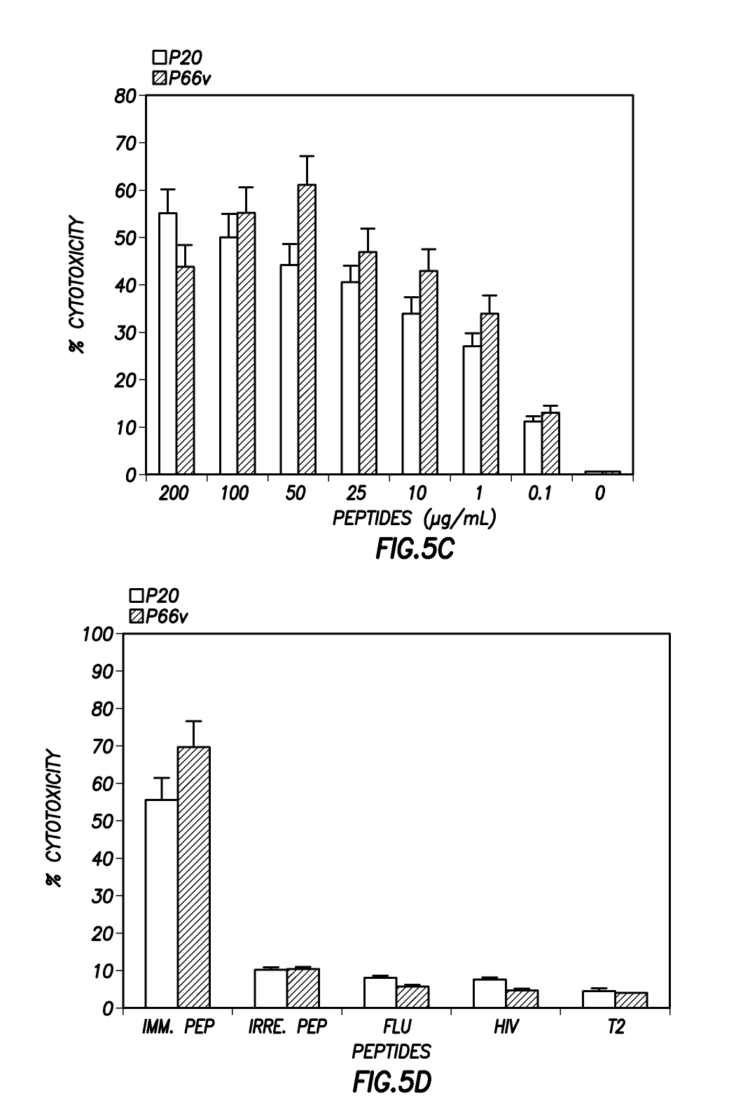

Using limiting-dilution assay, we obtained three T-cell clones (T4, T12 and T16; FIG. 5A) from a P20-specific T-cell line and five T-cell clones (T6, T19, T23, T26 and T27; FIG. 5B) from a P66v-specific T-cell line generated from patients with MM. These T-cell clones were identified based on secretion of IFN-γ in response to antigen stimulation. After expansion and further selection, we chose clone T16 (P20-CTL) and clone T6 (P66v-CTL) for further functional studies.

We assessed the cytotoxic activity of the clones against T2 cells pulsed with DKK1 or control peptides. First, we examined the cytolytic activity of the T-cell clones against T2 cells pulsed with different concentrations of DKK1 peptides and demonstrate a dose-dependent response (FIG. 5C). Second, we show that these T-cell clones lysed T2 pulsed with the specific DKK1 peptides P20 or P66v (P<0.01, compared with control peptides) but not unpulsed T2 cells or T2 cells pulsed with irrelevant DKK1 or control (Flu-matrix and HIV-pol) peptides (FIG. 5D), further confirming the specificity of the T-cell clones.

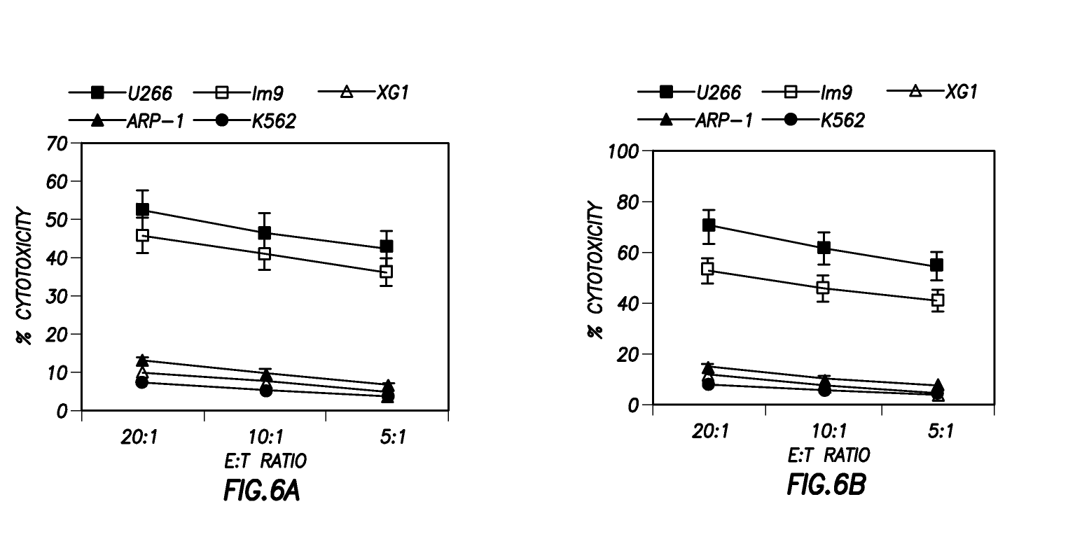

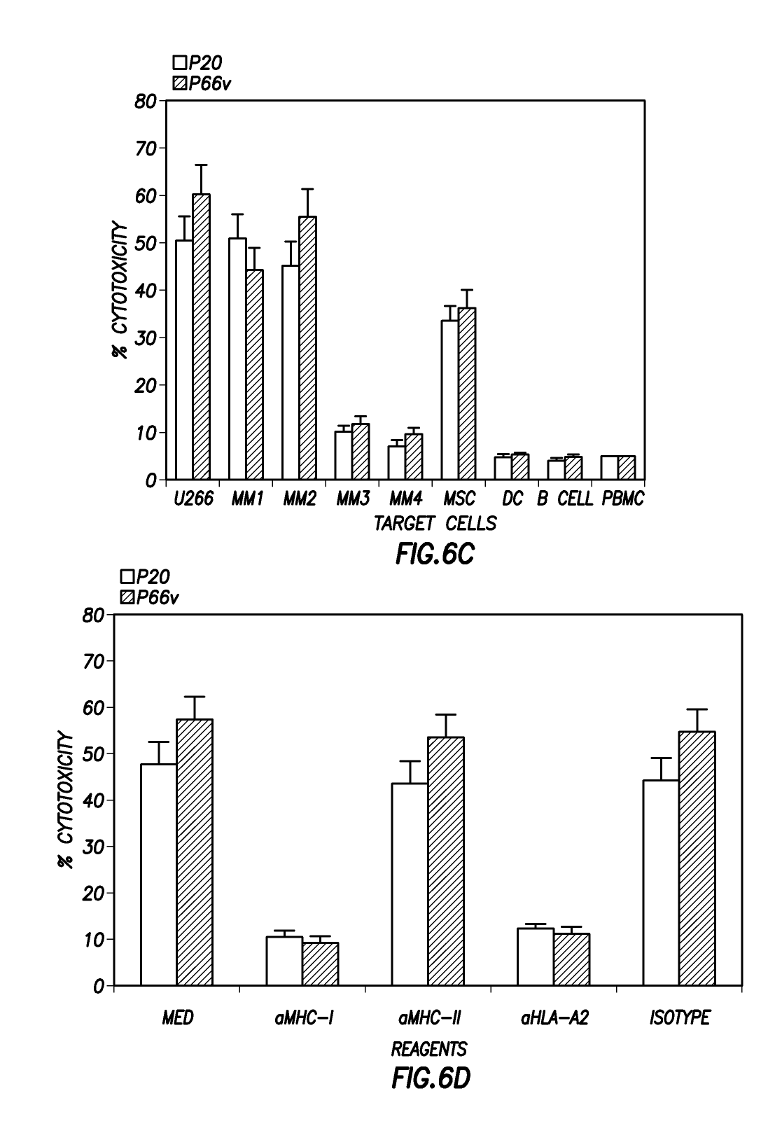

Next, we examined the cytolytic activity of the T-cell clones against myeloma cells, including HMCLs and primary myeloma cells isolated from patients with MM. As shown in FIGS. 6A and 6B, the T-cell clones effectively lysed DKK1⁺/HLA-A*0201⁺HMCLs U266 and IM-9 cells, but not DKK1⁻/HLA-A*0201⁺XG1 cells nor DKK1⁺/HLA-A*0201⁻ ARP-1 and MM.1S cells. No killing was observed against K562 cell line, excluding the possibility that NK cells contributed to the cytotoxicity. Furthermore, the T-cell clones efficiently killed DKK1⁺/HLA-A*0201⁺ primary myeloma cells from patients #1 and #2 but not myeloma cells from two DKK1⁺/HLA-A*0201⁻ patients (#3 and #4) (FIG. 6C). Altogether, these results demonstrate that the T-cell clones were not only able to lyse DKK1 peptide-pulsed T2 cells but also myeloma cells including primary myeloma cells from HLA-A*0201⁺ patients with MM, further confirming our findings that the DKK1 peptides are naturally presented in the context of HLA-A*0201 molecules by primary myeloma cells and shared among patients.

To determine MHC restriction of the T cell-mediated cytotoxicity, we evaluated the inhibitory effects of anti-MHC mAbs. As shown in FIG. 6D, mAbs against HLA-ABC or HLA-A*0201 significantly inhibited (70-80% inhibition) T cell-mediated cytotoxicity against peptide-pulsed T2 cells (P<0.01, compared with medium control). No inhibitory effect was observed with mAb against HLA-DR and isotype control IgG. The results indicate that the cytotoxicity was attributed to MHC class I and more specifically, HLA-A*0201-restricted CD8⁺ CTLs.

Finally, we examined whether the T cells were cytolytic to normal hematopoietic cells. In these experiments, autologous mature DCs, purified blood B cells (using anti-CD19 antibody-coated microbeads) and PBMCs, and MSCs (DKK1-expressing cells) from HLA-A*0201⁺ individuals were used as target cells. As shown in FIG. 6C, the T-cell clones did not kill DKK1⁻ DCs, B cells or PBMCs but lysed DKK1⁺MSCs, although the cytolytic activity against MSCs was weaker than that against myeloma cells.

### Cytotoxicity of the T-Cells was Mediated Via the Perforin Exocytosis Pathway

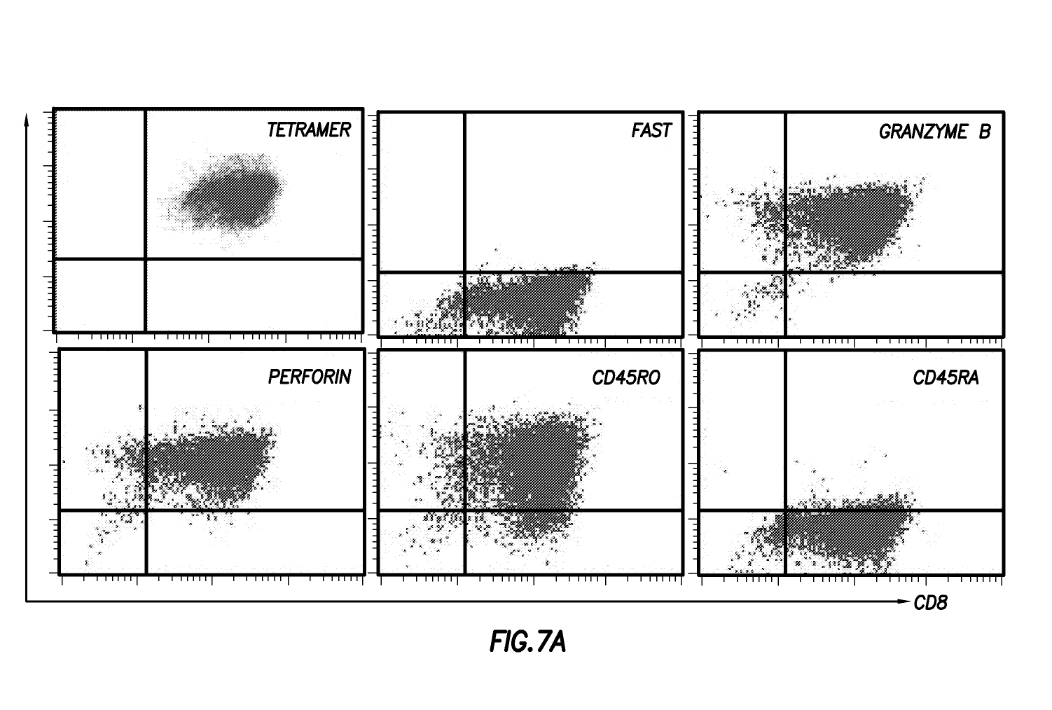

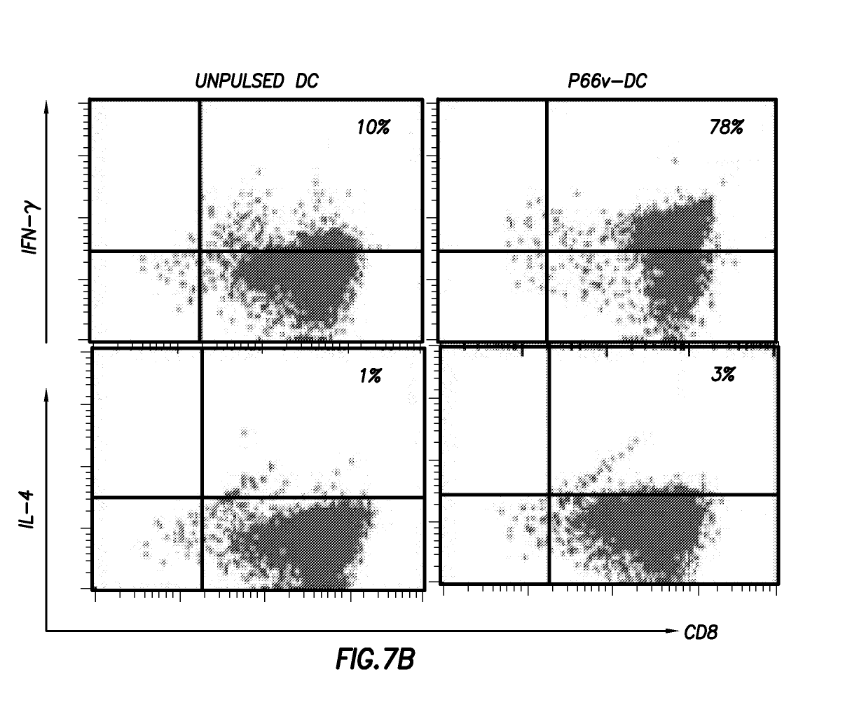

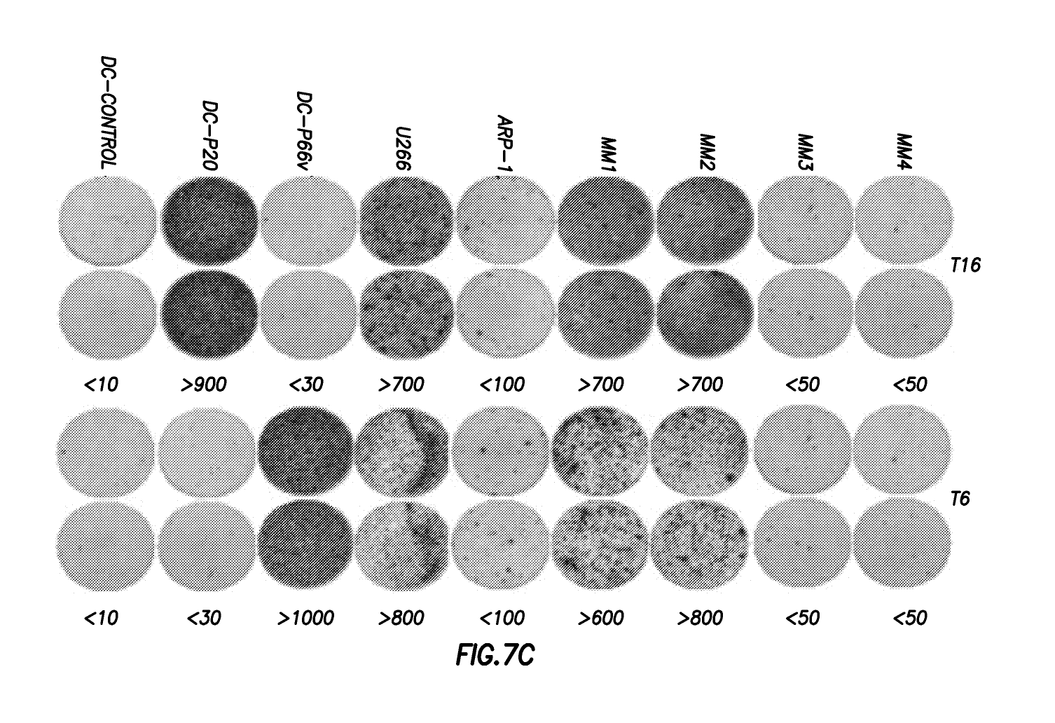

Flow cytometry analysis was used to examine the expression of granzyme, perforin and Fas ligand (FasL) by the T-cell clones. As shown in FIG. 7A, it seems that the T-cell clones killed their target cells via the perforin/granzyme pathways, because they expressed high levels of perforin and granzyme B but not FasL. The T-cell clones also expressed CD45RO, but not CD45RA, indicating that they were memory effector cells. de Jong, R., et al., Human CD8+ T Lymphocytes can be Divided Into CD45RA+ and CD45RO+ Cells With Different Requirements for Activation and Differentiation. J Immunol, 1991, 146:2088-2094; Merkenschlager, M., et al., Evidence for Differential Expression of CD45 Isoforms by Precursors for Memory-Dependent and Independent Cytotoxic Responses: Human CD8 Memory CTLp Selectively Express CD45RO (UCHL1), Int Immunol. 1989, 1:450-459; Dutton, R. W., et al., T Cell Memory, Annu Rev Immunol. 1998, 16:201-223.

### Expression and Production of IFN-γ by the T-Cells

Two independent methods were used to examine the cytokine expression profiles of the T cells. FIG. 7B shows a representative experiment of intracellular cytokine staining for IFN-γ and IL-4 expression in the P66v-specific T-cell clone. Upon restimulation with DCs pulsed with P66v peptide, but not with unpulsed DCs, high portion (80%) of the T cells expressed IFN-γ. IL-4-expressing T cells were very few (3%). To detect cytokine secretion, an ELISPOT assay was used to enumerate IFN-γ-secreting cells. After restimulation with DCs pulsed with DKK1 peptides, or with DKK1⁺/HLA-A*0201⁺ U266 and primary myeloma cells, large numbers of IFN-γ-secreting cells were detected (FIG. 7C). Other stimulatory cells, such as unpulsed DCs, and DKK1^{±}/HLA-A*0201⁻ ARP-1 or primary myeloma cells did not increase the number of IFN-γ-secreting cells. Taken together, the T-cell clones expressed IFN-γ, but not IL-4, and were thus the type-1 CD8⁺ T cells. Romagnani, S., Human TH1 and TH2 Subsets: Doubt No More, Immunol. Today. 1991, 12:256-257; Romagnani, S., The Th1/Th2 Paradigm and Allergic Disorders, Allergy. 1998, 53:12-15.

### EXAMPLE 2

### Effects of Adoptively Transferred DKK1 Peptide-Specific T cells in Eradicating Established Myeloma in SCID-hu Mouse Model

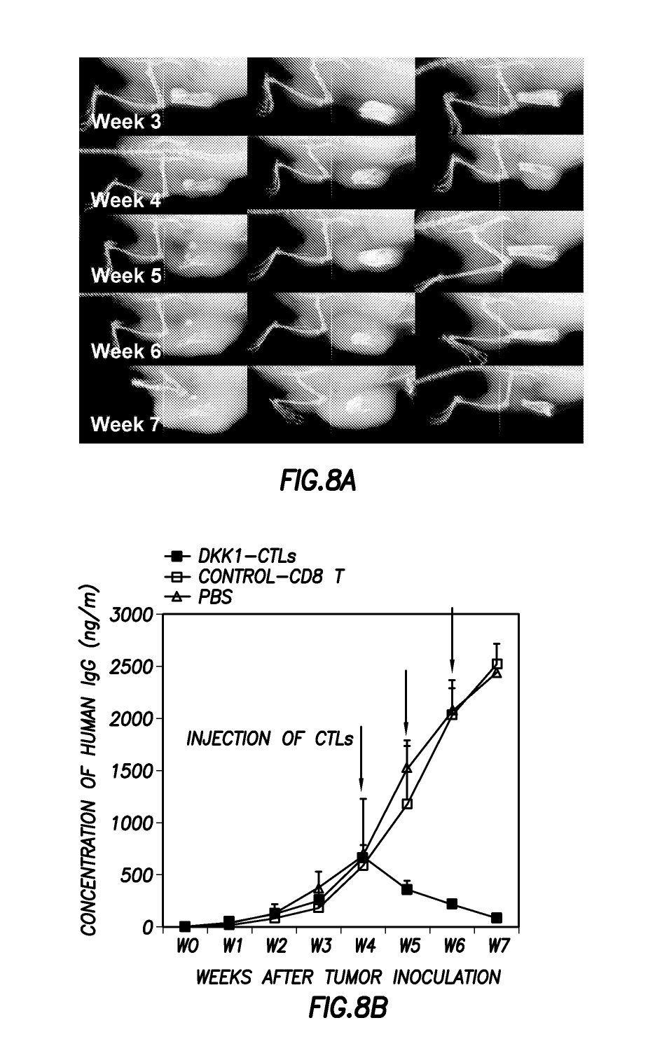

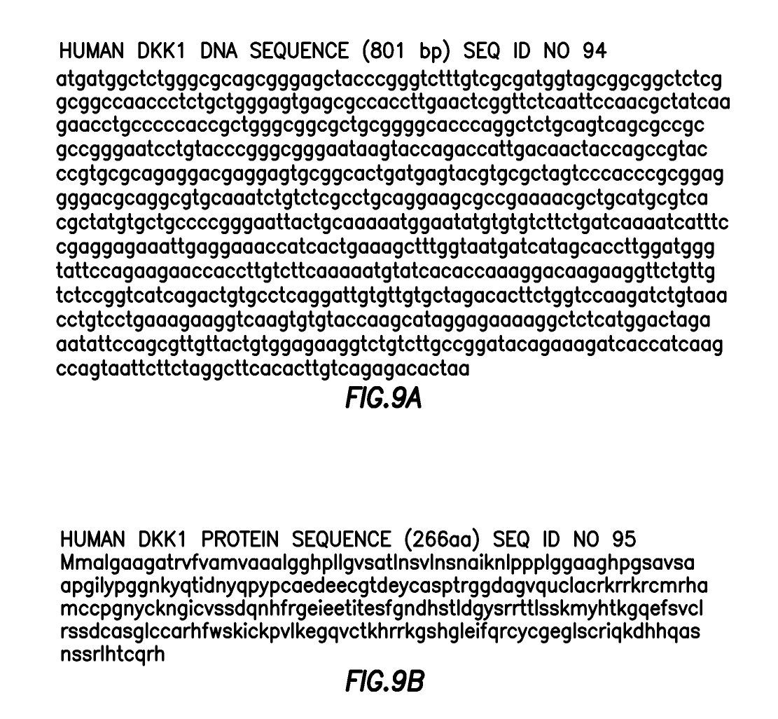

To examine the in vivo efficacy of the DKK1 peptide-specific cytotoxic T lymphocytes (CTLs) in controlling myeloma cells, we established primary myeloma-SCID-hu mouse model that contains human hematopoietic system or human bone marrow microenvironment. This system, in which primary myeloma cells grow, and interact with and are dependent on the human bone marrow microenvironment, offers the best representation of clinical myeloma. SCID mice were first implanted subcutaneously with fetal human bone chips and allowed to be vascularized. After that purified primary myeloma cells were injected into the human bone and mice developed myeloma, evident by the appearance and increase in serum of human M-protein secreted by the human myeloma cells. Once myeloma established in the mice, treatment with adoptive transfer of human DKK1 peptide-specific CTLs began; mice (3 per group) received two intravenous injections of either (5×10⁶ cells) DKK1 peptide-specific CTL (P66v-specific T-cell clone) or purified CD8⁺ T cells, or PBS (untreatment). As shown in FIGS. 8A and 8B, injection of DKK1 peptide-specific CTLs effectively controlled or eradicated the myeloma cells, whereas mice receiving control CD8⁺ T cells or PBS developed big tumor burdens. Hence, these results clearly demonstrate the ability of DKK1 peptide-specific CTLs to kill human myeloma cells in vivo.

### EXAMPLE 3

### DKK1 Protein and DNA Vaccines

Murine plasmacytoma cell lines MOPC-21 and MOPC-315 were obtained from the American Type Culture Collection (ATCC; Rockville, Md.), and cultured in DMEM complete medium (DMEM supplemented with 10% heat-inactivated fetal bovine serum, 1% penicillin/streptomycin, and 2 mM L-glutamine) (Gibco BRL, Gaithersburg, Md.). Female BALB/c mice, 6 to 8 weeks old, will be purchased from Jackson Laboratories (Bar Harbor, Me.). Mice are maintained in an American Association of Laboratory Animal Care-accredited facility, and studies have been approved by the Institutional Animal Care and Use Committee of the University of Texas M. D. Anderson Cancer Center.

### Construction of Mouse DKK-1 DNA Vaccine

The mouse DKK-10RF (signal peptide excluded) will be cloned into a pCMVE-derived vector by the XhoI and SmaI sites. The protein product will be a fusion protein with Df2beta in N-terminal and DKK-1 in C-terminal. The DNA construct will be injected into mouse as a DNA vaccine.

### Expression and Purification of Human and Mouse DKK-1 Proteins

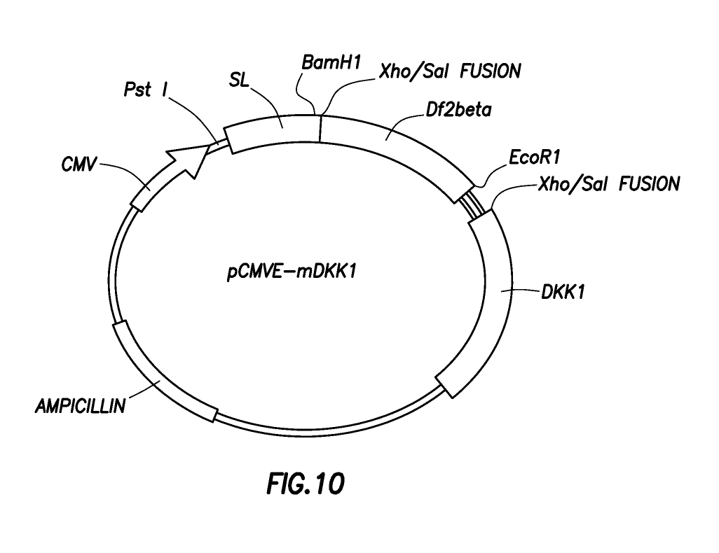

The human and mouse DKK-1 will be fused with a C-terminal Flag-tag and cloned into pcDNA3.1 vector to construct pcDNA3.1-hDKK-1-flag and pcDNA3.1-mDKK-1-flag constructs. See FIG. 10. The constructs will be transfected into 293T cells to produce human and mouse DKK-1, respectively. The culture supernatant will be collected to purify secreted DKK-1-flag proteins. The DKK-1-flag protein will be purified by anti-flag-IgG agarose beads and eluted by 3xflag peptide.

### In Vivo Immunizations and Tumor Protection Experiment

Each experiment includes four groups of mice (n=5) and will be repeated three times. Vaccinations consisted of DKK1 DNA plasmid or recombinant protein vaccines. Control mice will receive injections of phosphate-buffered saline (PBS) or DNA expressing chemokine defensin without DKK gene.

For DNA vaccine mice will be immunized with the Helios Gene Gun System (Bio-Rad, Hercules, Calif.) with 1-2 μg of plasmid DNA three times per 2 weeks as previously described. Biragyn, A., et al., 1999, Genetic Fusion of Chemokines to a Self Tumor Antigen Induces Protective, T-cell Dependent Antitumor Immunity, Nat. Biotechnol. 17:253. For recombinant DKK1 protein vaccine, mice will be immunized s.c. with 5 μg DKK1 protein mixed with 50 μg CpG ODN-1826 as adjuvant three times per 2 weeks.

Two weeks after the last immunization, mice will be challenged s.c. with 1×10⁶ MOPC-21 tumor cells and followed for survival. Differences in survival between groups are determined by nonparametric log-rank test (BMDP Statistical software, Los Angeles, Calif.). The p values refer to comparisons with the group immunized with PBS or control plasmid.

### Detection of Anti-DKK1 Antibodies

To evaluate antigen-specific antibody production, an enzyme-linked immunoabsorbent assay (ELISA) is used to measure titers of anti-DKK1 antibodies.

### Therapy of Established Tumor with DNA Vaccine

Six- to 8-wk-old female BALB/c mice (5/group) will be challenged s.c. with 1×10⁶ MOPC-21 tumor cells. On days 1, 4, 8, and 18, these mice are gene-gun immunized with DNA plasmid (containing 1-2 μg of DNA/immunization), or immunized s.c. with 5 □g recombinant DKK1 protein mixed with 50 μg CpG, and mice followed for tumor progression.

### Adoptive Transfer Experiments

BALB/c mice will be gene-gun immunized with 1-2 μg of DNA plasmid or 5 mg of recombinant DKK1 protein twice biweekly, and splenocytes and sera will be removed 10 days after the last immunization. Ten BALB/c mice per group will be s.c. injected in saline with 1×10⁶ MOPC-21 tumor cells/mouse mixed with sera from immune or mock-treated mice, or i.v. injected with 2×10⁷ splenocytes, and mice will be followed for tumor progression.

### In vivo T-Cell Subset Depletions

In vivo antibody depletions start 2 weeks after vaccination by treatment with three intraperitoneal doses of 400 μg anti-CD8 mAb 2.43, anti-CD4 mAb GK1.5 (both Protein G purified ascites), or normal rat IgG (Sigma). Treatments will be administered every other day, starting 2 weeks after the last immunization, and prior to tumor challenge. Depletion of lymphocyte subsets will be assessed 1 and 2 weeks after final treatment, using flow cytometry analysis. Splenocytes from normal mice treated with these mAbs will be assessed in parallel. Kwak, L. W., et al., Vaccination With Syngeneic, Lymphoma-Derived Immunoglobulin Idiotype Combined With Granulocyte/macrophage Colony-Stimulating Factor Primes Mice for a Protective T-Cell Response, Proc. Natl. Acad. Sci. USA 93, 10972-10977 (1996).

### Assays for IFN-γ and IL-4

Immune responses in immunized animals will be measured as a function of IFN-□ production using spleen cells and two related assays, ELISPOT and intracellular staining. In the ELISPOT assay, splenocytes will be tested for cytokine release following an overnight (18-20 h) protein (10 μg/ml) activation step. Intracellular staining of IFN-γ or IL-4 will be performed using a Cytofix/Cytoperm kit (BD Biosciences) according to the manufacturer's instruction. T cells are stimulated with 50 ng/mL PMA and 500 ng/mL ionomycin for 6 hours in the presence of 1 μl/mL Golgiplus to inhibit cytokine secretion. Activated T cells will be stained with FITC-labeled anti-CD3, CD4, or CD8, followed by fixation and permeabilization. Cells will be resuspended in perm/wash solution and stained with PE-labeled anti-IFN-γ or IL-4 mAbs (BD Pharmingen). After washing, cells will be harvested and analyzed.

### Cytotoxicity Assay

The standard ⁵¹Cr-release assay will be performed to examine the cytotoxicity of T cells as described previously. Target cells include Balb/c plasmacytoma cells MOPC-21, MOPC-315, MPC-11, HOPC-1F/12, and lymphoma cell A20, DC pulsed with DKK1 protein or HEK293 transfected with DKK1 gene. Splenocytes of mice from each group will be pooled and cultured with irradiated MOPC-21 cells for 5 days. After culture, T cells will be harvested and incubated with ⁵¹Cr-labeled target cells (10⁴ cells/well) at different effector-to-target cell ratios. Normal B cell and DCs from Balb/c mice will be used as control target cells. After a 4-hour culture, 50% of the supernatants will be collected, and radioactivity will be measured. Percent specific lysis is calculated using the following formula: percent specific lysis=(experimental counts−spontaneous counts)/(maximal counts−spontaneous counts).

### EXAMPLE 4

### DKK1 is Expressed in Lymphoma, Breast Cancer and Prostate Cancer Cell Lines

Lymphoma cell lines used included BJAB, RL, Daudi, CA46, SP53, Mino, Jeko-1, and Grant-519. U266 and PBMC (normal cells) were used as control. Breast cancer cell lines used included MCF-7, MDA-MB-231, MDA-MB-468. Prostate cancer cell lines used included PC-3, Lncap-LN3, and PC3M-LN4. Lymphoma and prostate cancer cell lines were maintained in RPMI-1640 medium (Fisher Scientific, Herndon, Va.) supplemented with 10% heat-inactivated fetal bovine serum (Atlanta Biologicals, Lawrenceville, Ga.), 1% penicillin/streptomycin, and 2 mM L-glutamine (Gibco BRL, Gaithersburg, Md.). Breast cancer cell lines were cultured in Leibovitz's L-15 (ATCC, Rockville, Md.) complete medium (L-15 supplemented with 10% heat-inactivated fetal bovine serum, 1% penicillin/streptomycin, and 2 mM L-glutamine (Gibco BRL, Gaithersburg, Md.).

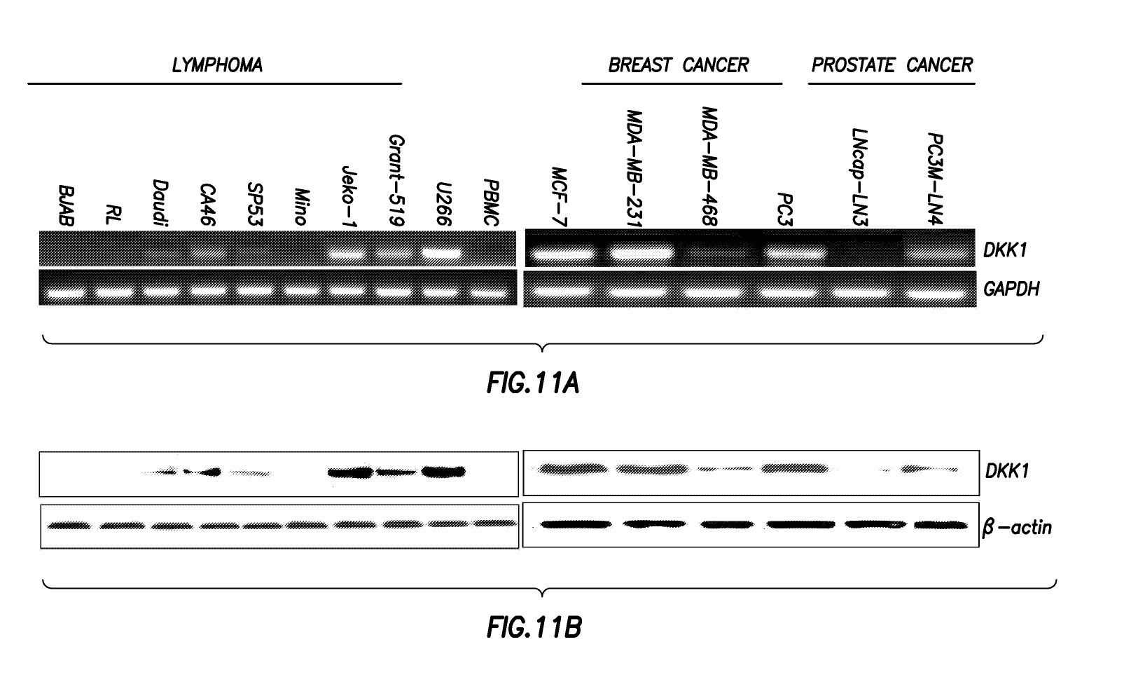

RT-PCR and Western blot analyses were used to examine the expression of DKK1 in normal human tissues and lymphoma cells, breast cancer cells and prostate cancer cells. DKK1 mRNA was detected in 6 out of 10 lymphoma cell lines, 3 breast cancer cell lines, and 2 out of 3 prostate cancer cell lines. (FIG. 11A). Similarly, DKK1 protein was detected in 6 out of 10 lymphoma cell lines, 3 breast cancer cell lines, and 2 out of 3 prostate cancer cell lines. (FIG. 11B).

### Cytotoxicity Assay

The standard 4-h ⁵¹Cr-release assay was performed to measure cytolytic activity of the T cells with target cells including lymphoma, breast cancer, and prostate cancer cell lines. Target cells were incubated with 100 μCi of ⁵¹Cr-sodium chromate for 1 h, washed extensively, seeded (1×10⁴ cells/well) into 96-well U-bottom plates in T-cell medium, and cocultured for 4 h with various numbers of T cells. All assays were performed in triplicates. Results are expressed as mean percentage of ⁵¹Cr release calculated as follows: [(sample counts−spontaneous counts)/(maximum counts−spontaneous counts)]×100%. Spontaneous release was less than 20% of the maximum ⁵¹Cr uptake.

To determine whether the cytolytic activity was restricted by major histocompatibility complex (MHC) class I or II molecules, 20 μg/mL mAbs against HLA-ABC (W6/32) or HLA-A*0201 (BB7.2; both purchased from Serotec Ltd., Oxford, UK), HLA-DR (B8.12.2; Immunotech, Marseilles, France), or isotypic controls (Immunotech) were added to the cultures at the start of the assay.

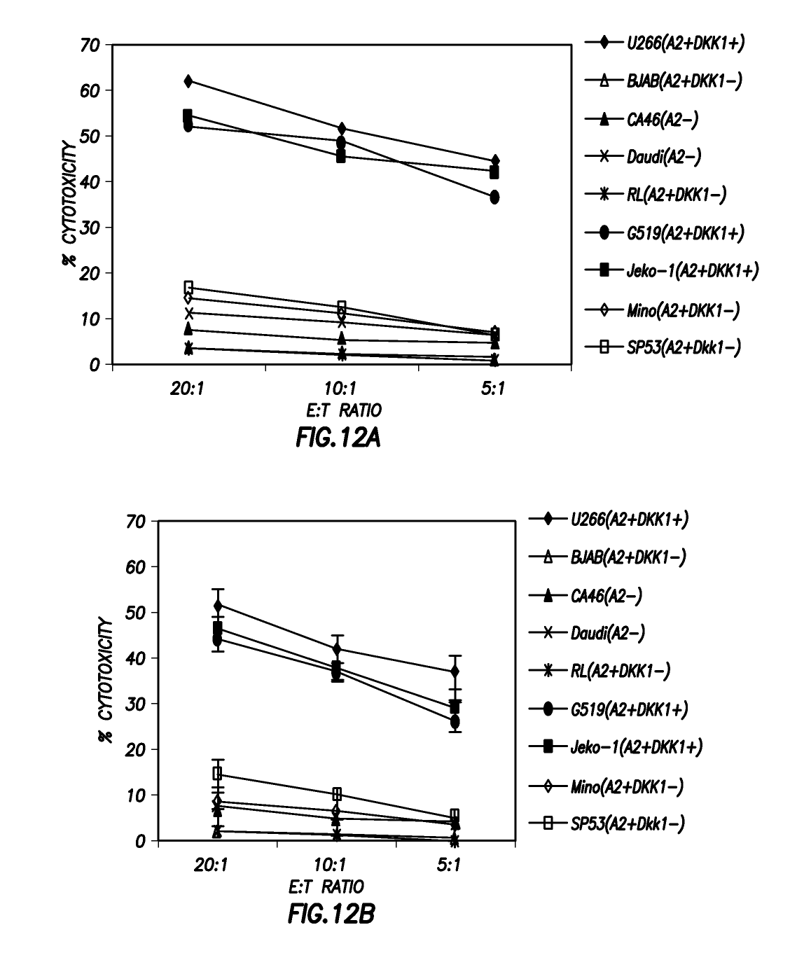

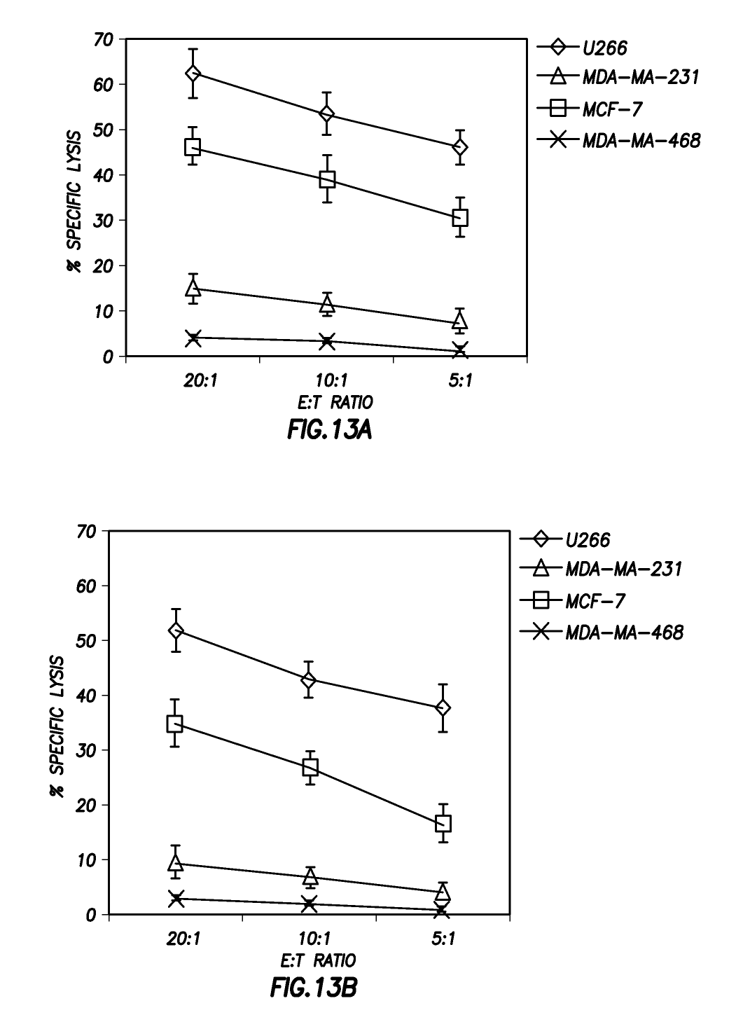

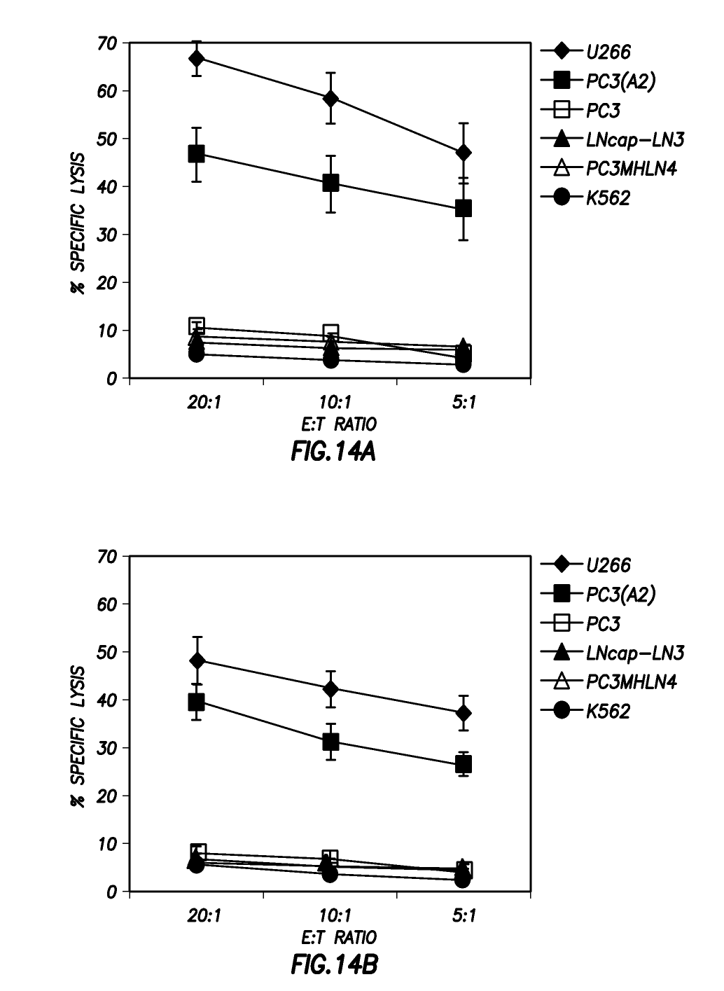

As exemplified by the results obtained with a T-cell line specific for P66v (FIG. 12A) or P20v (FIG. 12B), the T cells effectively lysed DKK1⁺/HLA-A*0201⁺ lymphoma cell lines Jeko-1 and Granta 519 cells, but not DKK1⁺/HLA-A*0201⁺BJAB, RL and Mino cells nor DKK1⁺/HLA-A*020⁻ CA46 and Daudi cells. Furthermore, the T-cell clones efficiently killed DKK1⁺/HLA-A*0201⁺ primary B-cell lymphoma cells from patients but not lymphoma cells from DKK17HLA-A*0201⁺ patients. HLA-ABC or HLA-A*0201 blocking mAbs significantly inhibited T cell-mediated cytotoxicity against peptide-pulsed T2 cells (P<0.01, compared with medium control). No inhibitory effect was observed with mAb against HLA-DR and isotype control IgG. The results indicate that the cytotoxicity was attributed to MHC class I and more specifically, HLA-A*0201-restricted CD8⁺ CTLs. The CTLs did not kill DKK1⁻/HLA-A*0201⁺ DCs, B cells, or PBMCs, These results suggest that the CTLs recognized DKK1 peptides that are naturally processed and presented in the context of HLA-A*0201 molecules on lymphoma cells.

### EXAMPLE 5

### Effects of Adoptively Transferred DKK1 Peptide-Specific T Cells in Eradicating Lymphoma and Prostate Cancer in NOS-SCID Mouse Model

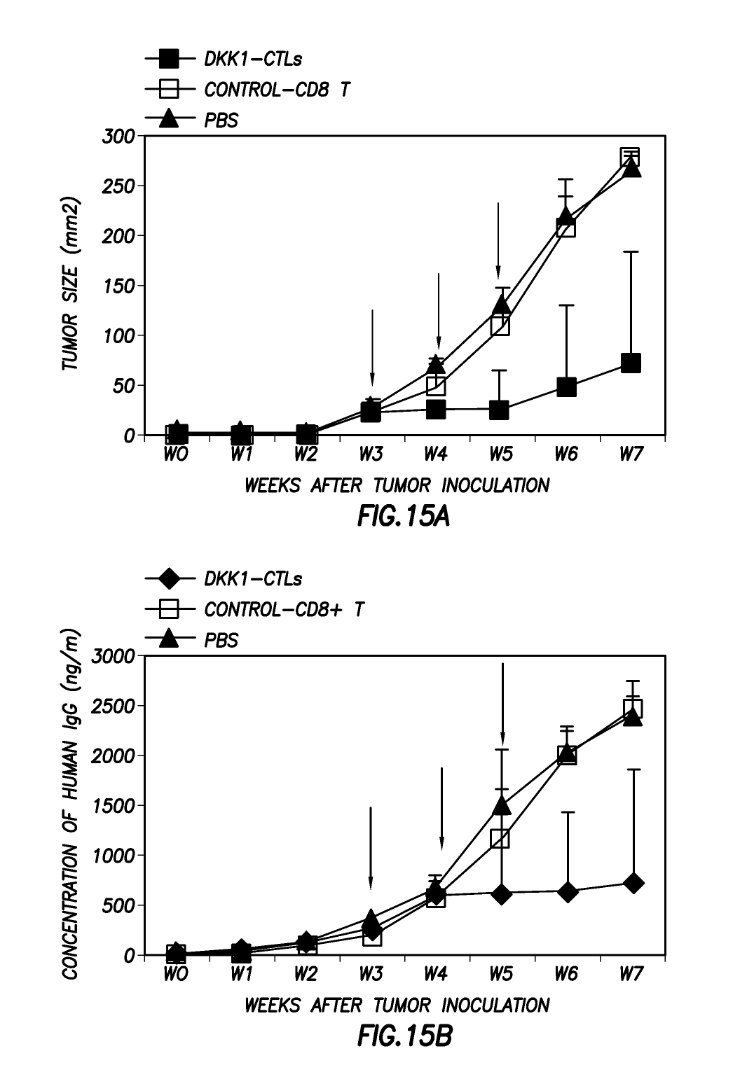

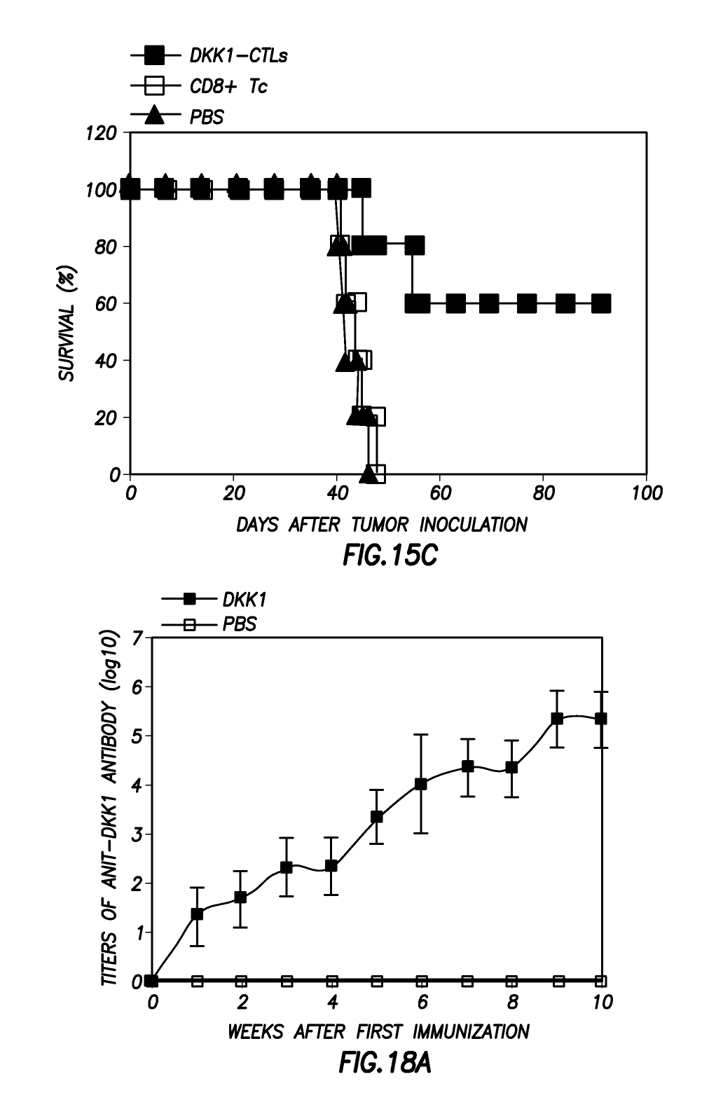

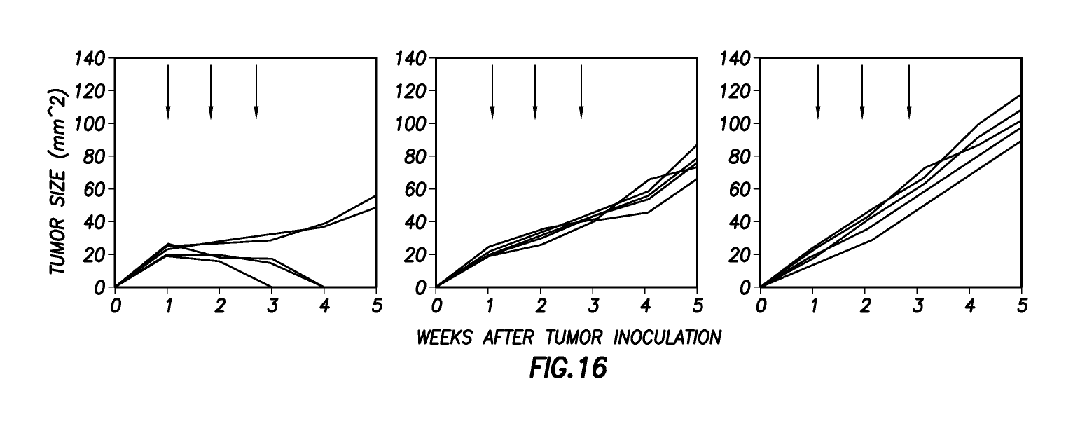

The in vivo efficacy of the DKK1 peptide-specific cytotoxic T lymphocytes (CTLs) in controlling lymphoma and prostate cancer cells was examined using a NOD-SCID mouse model. NOD-SCID mice, 6 to 8 weeks old, were purchase from The Jackson laboratory (Bar Harbor, Me.). To establish lymphoma or prostate cancer in the mice, each mouse was subcutaneously injected tumor cells with 5×10⁶. Once the tumor diameter was reached at 5 mm, treatment with adoptive transfer of human DKK1 peptide-specific CTLs began; mice (3 per group) received two intravenous injections of either (5×10⁶ cells) DKK1 peptide-specific CTL (P66v-specific T-cell clone) or purified CD8⁺ T cells, or PBS (untreatment). As shown in FIG. 15, injection of DKK1 peptide-specific CTLs effectively controlled or eradicated the lymphoma cells, whereas mice receiving control CD8⁺ T cells or PBS developed big tumor burdens. Also, as shown in FIG. 16, injection of DKK1 peptide-specific CTLs effectively controlled or eradicated the prostate cancer cells, whereas mice receiving control CD8⁺ T cells or PBS developed big tumor burdens. Hence, these results clearly demonstrate the ability of DKK1 peptide-specific CTLs to kill human lymphoma and prostate cancer cells in vivo.

### EXAMPLE 6

### Effects of Adoptively Transferred DKK1 Peptide-Specific T Cells in Eradicating Lymphoma in NOD-SCID and SCID-hu Mouse Models

To determine the in vivo antitumor activity, NOD-SCID and SCID-hu mice were used for lymphoma cell lines and primary lymphoma cells, respectively. Mice were treated with DKK1-specific CTLs after tumor established in NOD-SCID and SCID-hu mice. Control mice were treated with naïve CD8⁺ T cells or PBS alone. Tumor burden was measured according to levels of circulating human B2M, and survival rates were determined. Low levels (<50 ng/ml) of circulating human B2M were detected in group treated DKK1-specific CTLs, while high levels (≧150 ng/ml) of circulating human B2M were detected in control mice. In SCID-hu model, X-ray examination showed that established tumors were eradicated in 60% mice treated with DKK1-specific CTLs, while large tumor burdens were found in all control mice. In NOD-SCID model, 40% of mice survived with the treatment of DKK1-specific CTLs. TUNEL assay further confirmed that tumor cells were lysed by DKK1-specific CTLs not naïve CD8⁺ T cells. These results indicate that DKK1-specific CTLs are able to eradicate established, patient-derived primary B-cell lymphoma in the hosts and adoptive transfer of DKK1-specific CTLs may be used for B-cell lymphoma therapy.

### EXAMPLE 7

Primary mantle cell lymphoma cell lines used included ML4, ML5 and ML6. Follicular lymphoma cells used included (FL5). Myeloma cell line U266 was used as positive control.

### Expression of DKK1 Gene and Protein in Primary Mantle Cell Lymphoma and Follicular Lymphoma Cells

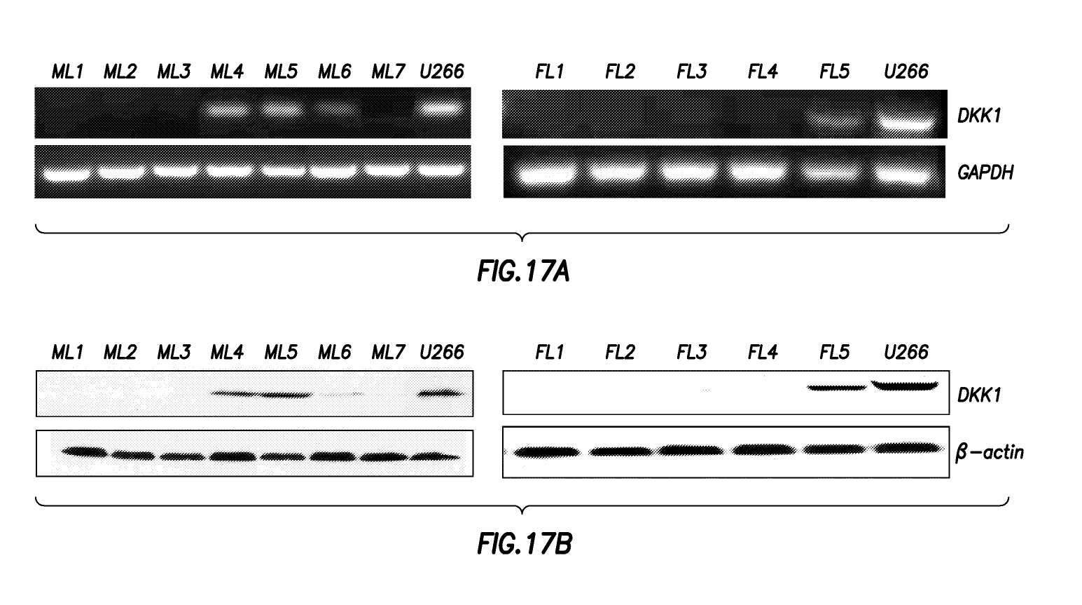

RT-PCR and Western blot analyses were used to examine the expression of DKK1 in primary mantle cell lymphoma cells and follicular lymphoma cells. FIG. 17A is RT-PCR showing DKK1 gene expression in some of primary mantle cell lymphoma (ML4, ML5 and ML6) and follicular lymphoma cells (FL5). FIG. 17B depicts western blots showing DKK1 protein expression in expression in some of primary mantle cell lymphoma (ML4, ML5 and ML6) and follicular lymphoma cells (FL5). Representative results of three independent experiments are shown.

### DKK1 Protein Vaccine can Protect Murine MM Challenge

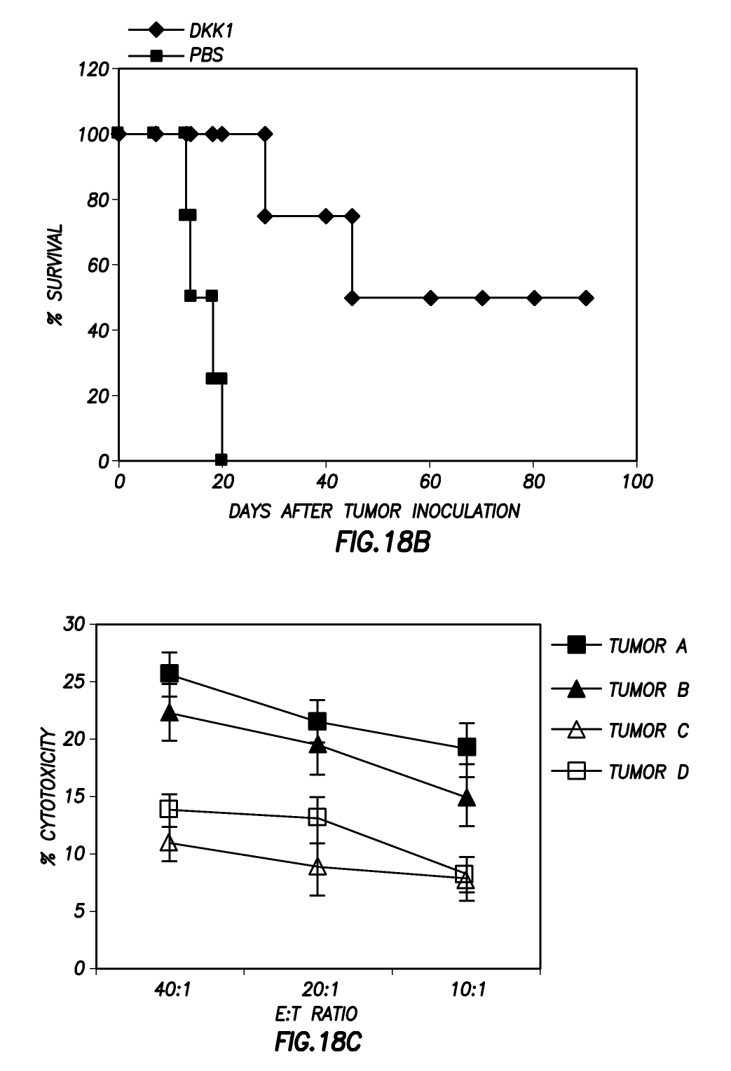

Balb/c mice (4 per group) were s.c. vaccinated three times with murine DKK1 protein (50 ug/mouse) at a 30-day interval followed by s.c. challenge with murine myeloma cell line tumor-A (1×10⁶ cells/mouse) after one weeks of third vaccination. Tumor burdens were measured twice every wk. Mice were euthanized when s.c. tumors reached 225 mm² or when mice became moribund. FIG. 18A is a graph depicting ELISA titers of anti-DKK1 antibody in the serum of immunized mice. Results shown in FIG. 18B are survival of mice that received DKK1 protein vaccinations and PBS control. Splenocytes were isolated from survival mice and pooled, and restimulated with irradiated tumor-A for 5 d. Results shown in FIG. 18C are cytotoxicity of CTLs against murine myeloma cell lines tumor-A, B, C and D. Representative results of three independent experiments are shown. The error bars in panels A and C indicate standard deviation of three independent experiments.

### DKK1 Peptides Vaccine can Protect Murine MM Challenge

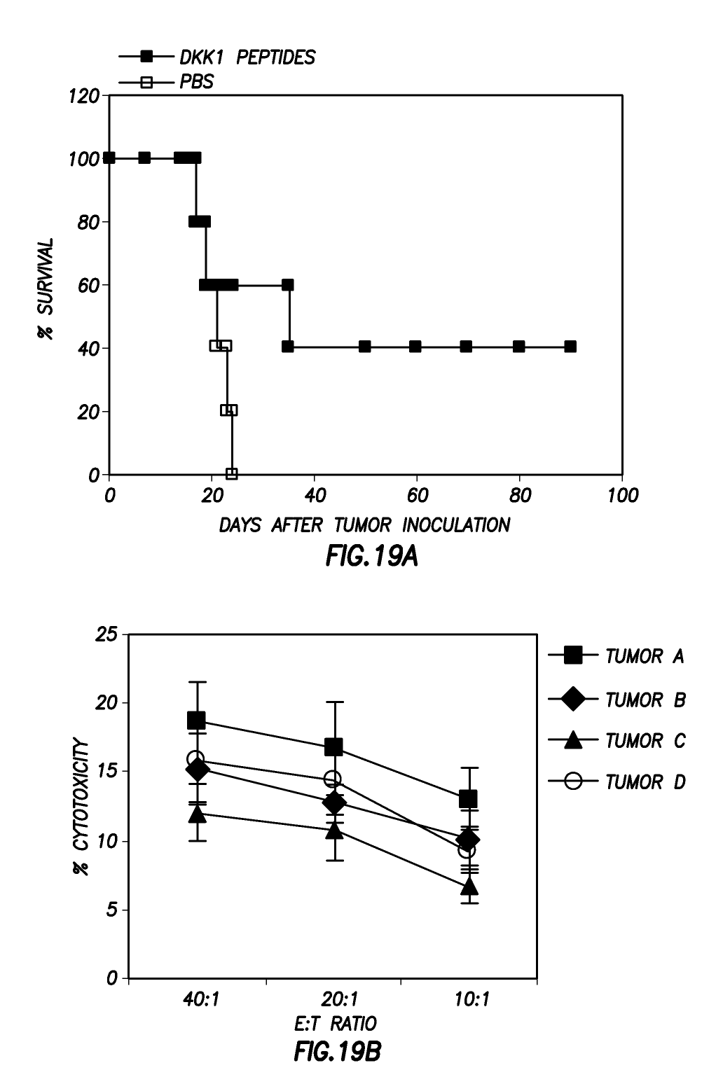

Balb/c mice (5 per group) were s.c. vaccinated three times with three DKK1 peptides (100 ug/mouse) at a 14-day interval followed by s.c. challenge with murine myeloma cell line tumor-A (1×10⁶ cells/mouse) after one week of third vaccination. Tumor burdens were measured twice every wk. Mice were euthanized when s.c. tumors reached 225 mm² or when mice became moribund. Results shown in FIG. 19A are survival of mice that received DKK1 peptides vaccinations and PBS control. Splenocytes were isolated from survival mice and pooled, and restimulated with irradiated tumor-A for 5 d. Results shown in FIG. 19B are cytotoxicity of CTLs against murine myeloma cell lines tumor-A, B, C and D. Representative results of three independent experiments are shown. The error bars in panels B indicate standard deviation of three independent experiments.

### DKK1 DNA Vaccine can Protect Murine MM Challenge

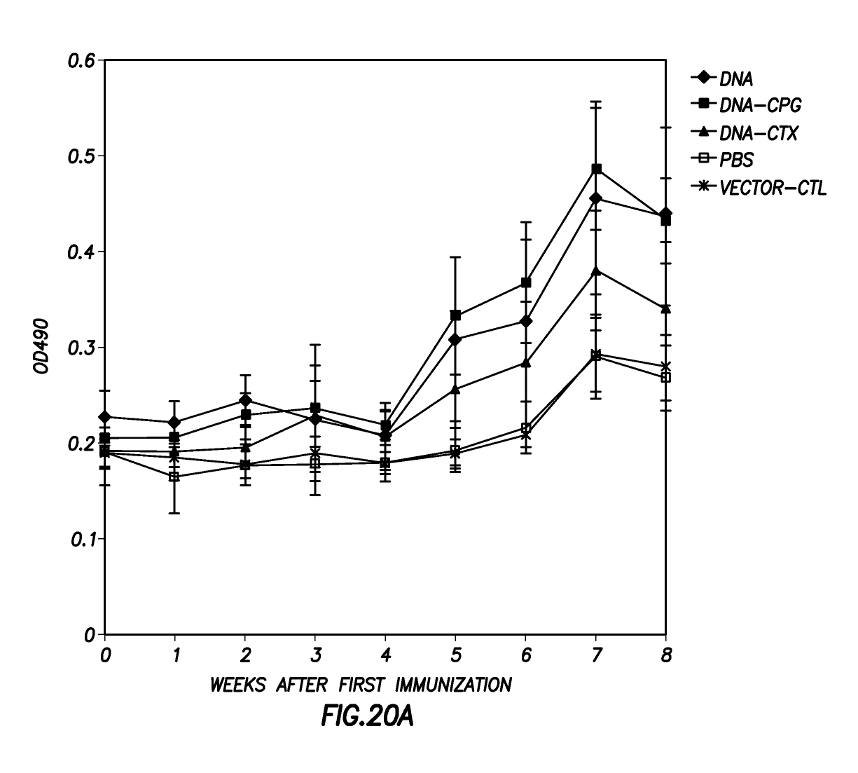

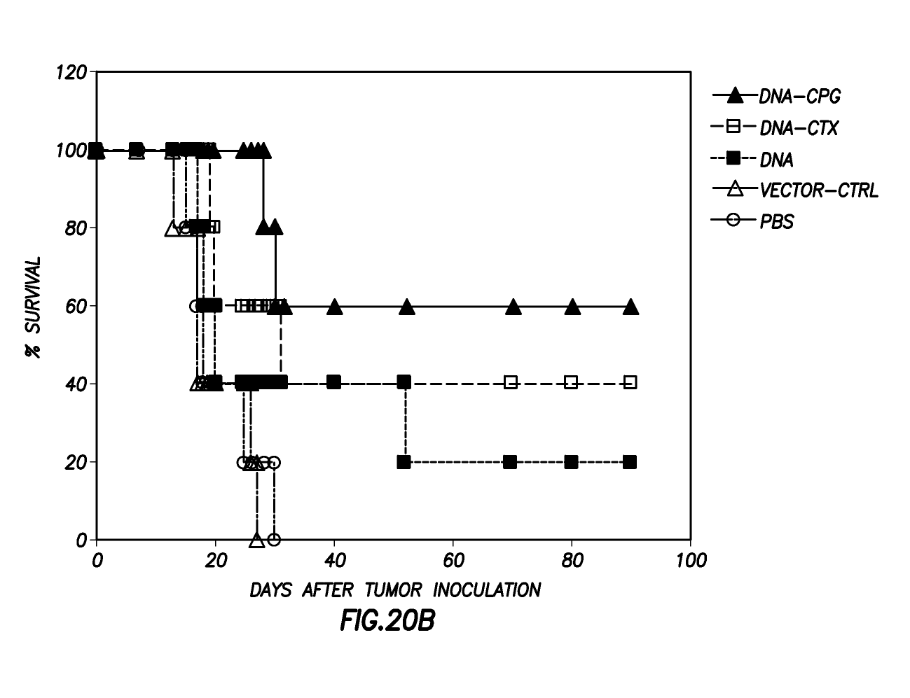

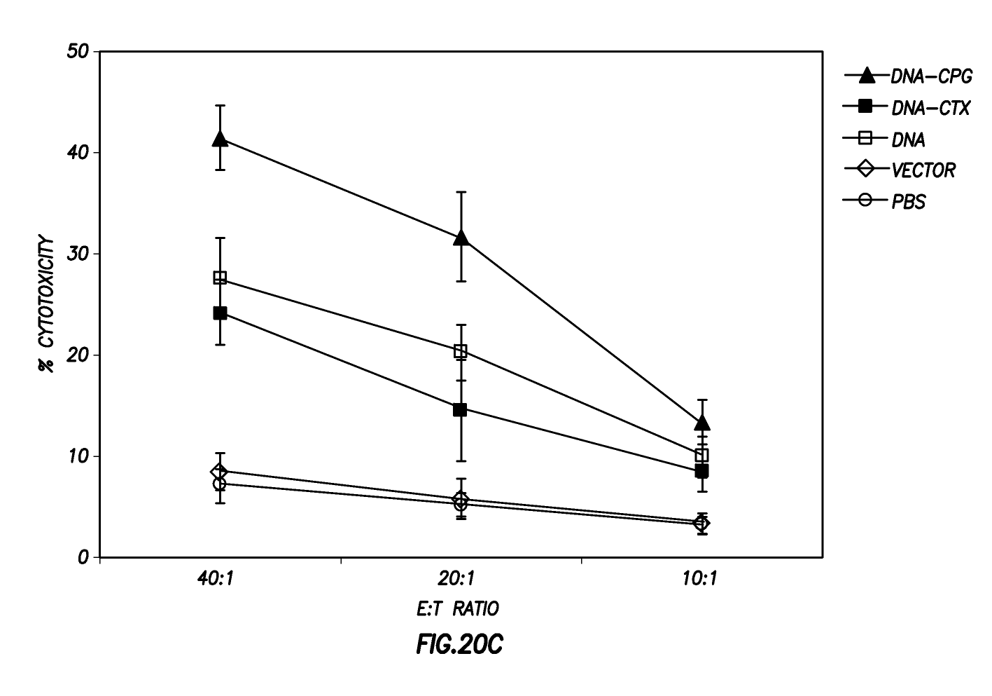

Balb/c mice (5 per group) were i.m. vaccinated three times with murine DKK1 DNA (50 ug/mouse) at a 14-day interval followed by s.c. challenge with murine myeloma cell line tumor-A (1×10⁶ cells/mouse) after one weeks of third vaccination. Tumor burdens were measured twice every wk. Mice were euthanized when s.c. tumors reached 225 mm² or when mice became moribund. FIG. 20A is a graph depicting ELISA titers of anti-DKK1 antibody in the serum of immunized mice. Results shown in FIG. 20B are survival of mice that received DKK1 DNA vaccinations and controls. Splenocytes were isolated from survival mice and pooled, and restimulated with irradiated tumor-A for 5 d. Results shown in FIG. 20C are cytotoxicity of CTLs against murine myeloma cell lines tumor-A. Representative results of three independent experiments are shown. The error bars in panels A and C indicate standard deviation of three independent experiments.

### DKK1 DNA Vaccine Induce CD4⁺ Tumor-Specific T-Cell Responses

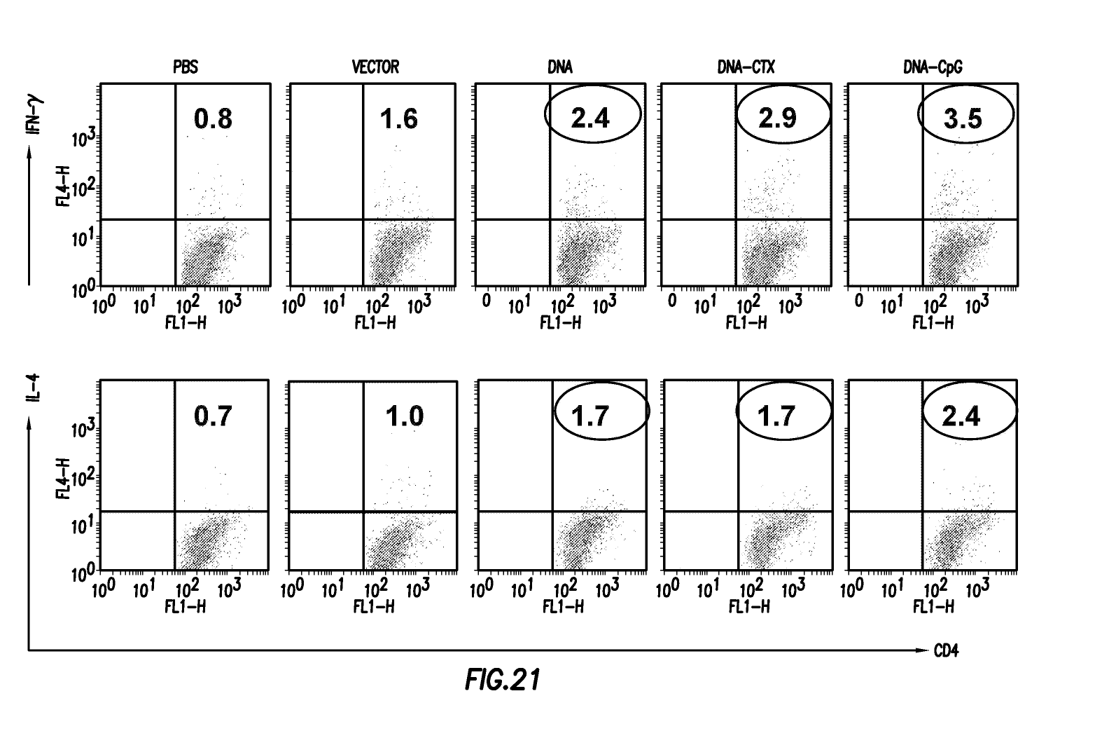

Balb/c mice (5 per group) were i.m. vaccinated three times with murine DKK1 DNA (50 ug/mouse) at a 14-day interval followed by s.c. challenge with murine myeloma cell line tumor-A (1×10⁶ cells/mouse) after one weeks of third vaccination. Splenocytes were isolated from survival mice and pooled, and restimulated with irradiated tumor-A for 5 d. Results shown in FIG. 21 are intracellular staining for CD4⁺ IFN-γ or IL-4-expressing T cells. Representative results of three independent experiments are shown. The numbers in quadrants plots presented are the percentages of IFN-γ or IL-4 positive staining CD4⁺ T cells.

### DKK1 DNA Vaccine Induce CD8⁺ Tumor-Specific T-Cell Responses

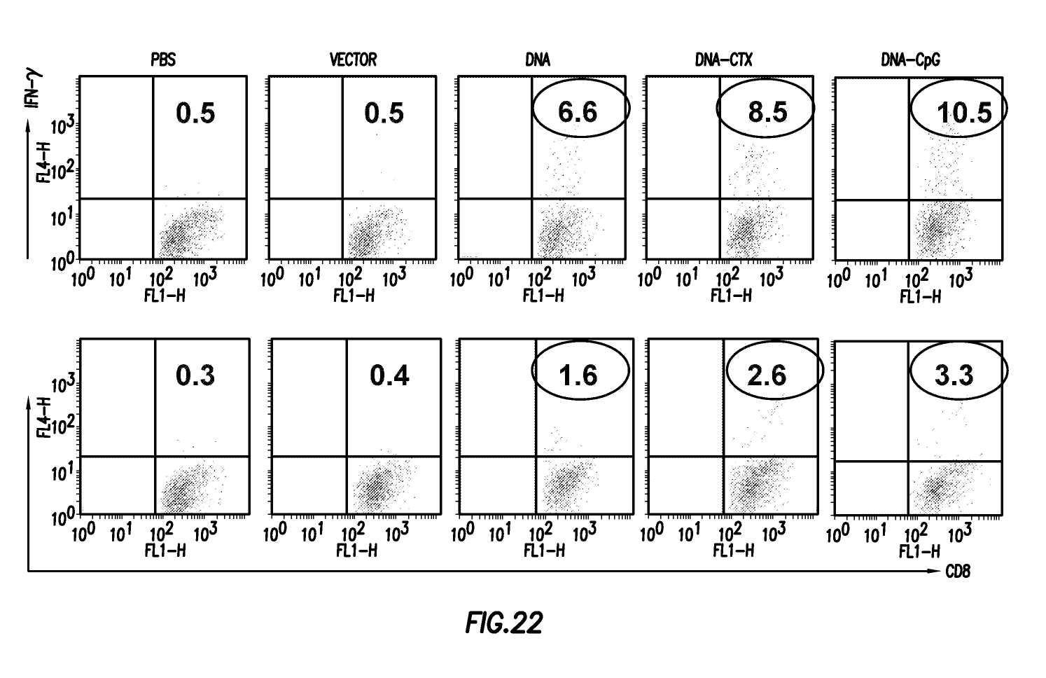

Balb/c mice (5 per group) were i.m. vaccinated three times with murine DKK1 DNA (50 ug/mouse) at a 14-day interval followed by s.c. challenge with murine myeloma cell line tumor-A (1×10⁶ cells/mouse) after one weeks of third vaccination. Splenocytes were isolated from survival mice and pooled, and restimulated with irradiated tumor-A for 5 d. Results shown in FIG. 22 are intracellular staining for CD8⁺ IFN-γ or IL-4-expressing T cells. Representative results of three independent experiments are shown. The numbers in quadrants plots presented are the percentages of IFN-γ or IL-4 positive staining CD8⁺ T cells.

We claim:

1. An isolated peptide comprising an amino acid sequence of SEQ ID NO. 1, SEQ ID NO. 2, SEQ ID NO. 3, or SEQ ID NO. 4.

2. A vaccine composition comprising at least one isolated peptide having at least 90% sequence identity to an amino acid sequence selected from the group consisting of SEQ ID NO. 1, SEQ ID NO. 2, SEQ ID NO. 3, and SEQ ID NO. 4, and a pharmaceutically acceptable carrier.

3. A vaccine composition comprising at least one isolated peptide having at least 10% identity to an amino acid sequence selected from the group consisting of SEQ ID NOS. 6-93 and a pharmaceutically acceptable carrier.

4. A vaccine composition comprising a DKK1 protein or DNA encoding a DKK1 protein and a pharmaceutically acceptable carrier.

5. A method of making a DKK1-specific cytoxic T lymphocyte or a DKK1-specific Th1 cell comprising stimulating autologous T cells in vitro with DKK1 peptide-loaded dendritic cells.

6. The method of claim 5 wherein the DKK1 peptide-loaded dendritic cells comprise at least one DKK1 peptide having at least 10% identity with an amino acid sequence selected from the group consisting of SEQ ID NOS. 1, 2, 3, 4 and 6-93.

7. A method comprising administering to a subject in need thereof a DKK1 vaccine comprising a DKK1 protein, a DKK1 peptide or DNA encoding a DKK1 protein.

8. The method of claim 7 wherein the DKK1 vaccine comprises at least one DKK1 peptide having at least 10% identity with an amino acid sequence selected from the group consisting of SEQ ID NOS. 1, 2, 3, 4 and 6-93.

9. The method of claim 7 wherein the DKK1 vaccine is administered in amount of about 0.1 to 500 mg/kg per day.

10. A method comprising administering to a subject in need thereof a therapeutically effective amount of at least one DKK1 peptide-specific cytotoxic T lymphocyte or DKK1 peptide-specific Th1 cell.

11. The method of claim 10 wherein the DKK1 peptide-specific cytotoxic T lymphocyte is capable of binding a DKK1 peptide having at least 10% identity with an amino acid sequence selected from the group consisting of SEQ ID NOS. 1, 2, 3, 4 and 6-93.

12. The method of claim 10 wherein the subject has a cancer.

13. The method of claim 12 wherein the cancer is multiple myeloma, a lymphoma, breast cancer or prostate cancer.

14. The method of claim 10 further comprising administering a second therapeutic agent.

15. The method of claim 10 wherein the DKK1 peptide-specific cytotoxic T lymphocyte or DKK1 peptide-specific Th1 cell is administered intravenously.
